(19) United States
(12) Patent Application Publication
(10) Pub. No.: US 2005/0059978 A1
(43) Pub. Date: Mar. 17, 2005

(54) Apparatus and methods for bone surgery

(76) Inventors: Eugene Sherry, Penrith (AU); Michael Egan, Blacktown (AU)

Correspondence Address: Russell D Orkin, 700 Koppers Building, 436 Seventh Avenue, Pittsburgh, PA, 15219-1818, US

(21) Appl. No.: 10/494,085

(22) PCT Filed: Nov. 1, 2002
(86) PCT No.: PCT/AU02/01482

(30) Foreign Application Priority Data
Nov. 2, 2001 (AU) PR 8657

(51) Int. Cl. A61F 5/00

(52) U.S. Cl. 606/87

(57) ABSTRACT

The surgeon grasps the jig by its handle and manipulates the head of the jig through the patient wound and onto the femoral neck. The head of the jig includes jig location means in the form of an elongate rod which acts as a spacer. The spacer has an end which abuts the trochanteric fossa so as to position the slot of the jig at the required position, which is between 5 mm and 25 mm, and most preferably 15 mm, from the trochantic fossa. Additional jig location means are provided by a surface adapted to receive a bone formation. This surface is provided by contours on the base of the head which are adapted to mate with contours of the femur. The slot is oriented generally perpendicularly to the elongate dimension of the rod. The slot functions as a surgical tool guide means which is positioned by the jig at the correct position for osteotomisation of the neck. Advantageously, osteotomisation takes place while the femoral head is still disposed within the acetabulum.

### FIELD OF THE INVENTION

The present invention relates to surgical apparatus and surgical methods and in particular those associated with osteotomological surgery on either humans or animals.

The invention has been developed primarily for use in hip replacement surgery and will be described hereinafter with reference to this application. However, it will be appreciated that the apparatus of the present invention is not limited to this particular field of use, but rather may be adapted for use with any bone structure and in relation to various types of surgery.

### BACKGROUND ART

Prior art hip replacement surgical techniques typically involve a surgeon firstly making a fairly large initial incision so as to reveal the hip joint. The surgeon then typically manipulates the leg of the patient to dislocate the hip bone. This often requires the application of significant force to effect dislocation. Excessive manipulation and application of force may cause collateral damage to the patient, possibly resulting in post operative pain and/or an extended healing time. The head of the femur is then cut off at the neck. A cavity is reamed into the hip to accept a prosthetic acetabular cup (for example a LINK T.O.P. Acetabular Cup) and a prosthetic stem (for example a LINK C.F.P. Hip Stem) is inserted into the femoral shaft. Typically the surgeon aligns the cutting and reaming tools by eye, possibly resulting in minor misalignments. Hence, once the prosthetics are installed, there may be visually imperceptible misalignments between the prosthetic acetabular cup and the prosthetic femoral head. This may result in problems such as misalignment of the leg, incorrect leg length and/or incorrect soft tissue tension. Additionally, in the long term, misaligned prosthetic components may wear more quickly, giving rise to aseptic loosening of components and potentially necessitating early repetition of the surgery.

Any discussion of the prior art throughout the specification should in no way be considered as an admission that such prior art is widely known or forms part of common general knowledge in the field.

### SUMMARY OF THE INVENTION

It is an object of the present invention to overcome or ameliorate at least one of the disadvantages of the prior art, or to provide a useful alternative.

According to a first aspect of the present invention there is provided a surgical jig for application to a bone, said jig including:
jig location means adapted to mate with bone formations so as to locate the jig in a predefined relationship with the bone; and
surgical tool guide means disposed relative to said jig location means such that, when said jig is mated with said bone formation, the surgical tool guide means is adapted to guide a surgical tool towards a predefined site on said bone formation.

According to a second aspect of the invention there is provided a surgical jig for application to a femur during hip replacement surgery, said jig including:
at least one lug adapted to mate with a bone formation on said femur so as to locate the jig in a predefined relationship with the femur; and
one or more slots disposed relative to said at least one lug such that, when said jig is mated with said femur, the one or more slots are adapted to guide a blade towards a predefined site on said bone formation.

Preferably the at least one lug is a rod disposed on a head of the jig so as to define an end adapted to engage a femoral bone formation and thereby position said one or more slots in a predefined relationship to said femoral bone formation.

Also preferably the jig further includes contours disposed on a base of the head, said contours matching, and being adapted to mate with, a femoral bone formation.

According to a third aspect of the invention there is provided a method of performing hip replacement surgery on a patient, said method including the steps of:
a) exposing a first femoral bone formation and a second femoral bone formation of the patient;
b) applying a jig having surgical tool guide means to the first femoral bone formation such that said surgical tool guide means is proximal the second femoral bone formation;
c) using the surgical tool guide means to guide a cutting tool so as to osteotomise the second bone formation; and
d) removing a severed femoral head from the patient's hip joint.

Preferably the first femoral bone formation is a greater trochanter and the second femoral bone formation is a femoral neck.

In the preferred embodiment the method also includes combinations of the following steps:
e) placing a guide wire into a femoral canal of a femoral shaft of the patient;
f) using the guide wire to guide a flexible reamer so as to bore an aperture into said femoral canal;
g) inserting a prosthetic stem into said aperture such that a first end of the stem protrudes from an end of the femoral shaft;
h) placing said first end into an acetabulum of the patient;
i) inserting spacers intermediate the end of the femoral shaft and the acetabulum;
j) using a spacer to determine reference points on an acetabulum of the patient;
k) using said reference points to provide guidance for reaming of the acetabulum;
l) placing a trial prosthetic head onto said first end m) placing a trial acetabular cup into the reamed actabulum;
n) placing said trial prosthetic head into said trial acetabular cup;
o) inserting spacers intermediate the end of the femoral shaft and the trial acetabular cup until a required fit between the trial head and the acetabulum is obtained; and
p) correlating the number or size of spacers with a required size of prosthetic femoral head and neck component.

According to a fourth aspect of the present invention there is provided a method of performing hip replacement surgery on a patient, said method including the steps of:
i) exposing a greater trochanter, and a femoral neck of the patient;
ii) osteotomising the femoral head;
iii) inserting a prosthetic stem or trial stem into a femoral shaft of the patient such that a first end of the stem protrudes beyond an end of the femoral shaft;
iv) positioning the first end into an acetabulum of the patient;
v) applying one or more spacers to the first end of the stem;
vi) using said one or more spacers to determine reference positions on the acetabulum; and
vii) using said reference positions to guide reaming of said acetabulum.

Preferably said one or more of said spacers include markings adapted to indicate said reference positions. Also for preference, step vii) includes determining a center of said reference positions and using said center as a center of reaming.

According to a fifth aspect of the present invention there is provided a method of performing hip replacement surgery on a patient, said method including the steps of:
A) exposing a greater trochanter, and a femoral neck of the patient;
B) osteotomising the femoral head;
C) inserting a prosthetic stem or trial stem into a femoral shaft of the patient such that a first end of the stem protrudes beyond an end of the femoral shaft;
D) inserting a prosthetic actabular cup or trial acetabular cup into a reamed acetabulum of the patient;
E) applying one or more spacers to the first end of the stem until a desired tissue tension in the patient's hip joint region has been obtained; and

F) correlating the number or size of spacers with a required prosthetic component size.

Preferably said required prosthetic component size relates to a neck length of a prosthetic femoral neck and head component. Also for preference, at least some of the spacers have a thickness corresponding to size increments between prosthetic components.

According to a sixth aspect of the present invention there is provided a method of performing hip replacement surgery on a patient, said method including the steps of:
A) exposing a greater trochanter, and a femoral neck of the patient;
B) osteotomising the femoral head;
C) inserting a prosthetic stem or trial stem into a femoral shaft of the patient such that a first end of the stem protrudes beyond an end of the femoral shaft;
D) inserting a prosthetic actabular cup or trial acetabular cup into a reamed acetabulum of the patient; and

E) applying one or more spacers to the first end of the stem so as to determine a required orientation of the acetabular cup relative to the stem.

According to a seventh aspect of the invention there is provided a spacer for use in hip replacement surgery, said spacer including:
a handle defining a first end; and
alignment means disposed at or adjacent to said first end, said alignment means being adapted to mate with a prosthetic acetabular cup such that, in use, the cup is positioned in a predefined relationship with reference to an acetabulum of a patient.

According to an eighth aspect of the invention there is provided an alignment handle for use in hip replacement surgery, said alignment handle including:
a body defining a distal end and a proximal end;
a gauge disposed at or proximate to said proximal end; and
a connector disposed at or proximate to said distal end for connection of a prosthetic acetabular cup,
   said gauge being adapted to indicate whether said prosthetic acetabular cup is in a predefined orientation with respect to at least a first plane in a referential system.

Preferably the referential system includes a local gravitational field.

According to a ninth aspect of the invention there is provided an alignment frame adapted for use with an alignment handle as described above, said alignment frame including:
a first end adapted for abutment against a planar surface so as to align a second end of said alignment frame with respect to said planar surface; and
engagement means disposed on said second end and adapted for engagement with said alignment handle.

Preferably the planar surface is a side of an operating table.

According to a tenth aspect of the invention there is provided a method for extracting an osteotomitised femoral head from a patient's hip joint, said method including one or both of the following steps:
a) drilling a hole into the femoral head, threading a self-tapping threaded member into said hole and exerting an outwardly directed force onto said threaded member which is, in turn, exerted onto the femoral head; and/or
b) wedging an end of a scoop under the femoral head and using said scoop as a lever so as to exert an outwardly directed force onto said femoral head.

According to an eleventh aspect of the invention there is provided a method for inserting a prosthetic acetabular cup into a patient's reamed acetabulum during hip replacement surgery, said method including the steps of:
a) mounting said prosthetic acetabular cup onto a cup alignment tool;
b) positioning the cup alignment tool such that the prosthetic acetabular cup is adjacent the reamed acetabulum;
c) orienting the cup alignment tool to ensure that the prosthetic acetabular cup is in an anatomically correct orientation for insertion into the reamed acetabulum; and
d) impacting the prosthetic acetabular cup into the reamed acetabulum.

According to another aspect of the invention there is provided a method for ascertaining a size of a prosthetic neck length during hip replacement surgery, said method including the steps of:
a) installing a prosthetic stem into a patient's femur such that an end of said stem projects from an end of the femur;
b) placing a trial head onto the end of the prosthetic stem;
c) using a spacer alignment tool to position a spacer intermediate the trial head and the end of the femur;
d) orienting the spacer alignment tool to ensure that the trial head is in an anatomically correct orientation;
e) testing whether the patient's leg length and/or tissue/muscle tension is anatomically correct;
f) repeating steps c), d) and e) with different spacer thicknesses or numbers of spacers until the patient's leg length and/or tissue tension and/or muscle tension is anatomically acceptable and taking note of the spacer thickness or number of spacers which gives the best result; and
g) correlating the spacer thickness or number of spacers determined in step f) with the required prosthetic neck length in accordance with a predefined correlation scheme.

### BRIEF DESCRIPTION OF THE DRAWINGS

Preferred embodiments of the invention will now be described, by way of example only, with reference to the accompanying drawings in which:

FIG. 1 is a perspective view of a jig according to the preferred embodiment of the present invention;

FIG. 2 is plan view of the jig shown in FIG. 1;

FIG. 3 is side view of the jig shown in FIG. 1;

FIG. 4 is front end view of the jig shown in FIG. 1;

FIG. 5 is a depiction of a greater trochanter, femoral neck and femoral head with markings illustrating the sites at which the three available cutting lines intersect the femoral neck and the manner in which the jig engages with the formations on the femur;

FIG. 6 is a perspective view of a spacer according to a preferred embodiment of the present invention;

FIG. 7 is a plan view of the spacer shown in FIG. 6;

FIG. 8 is a perspective view of a scoop according to a preferred embodiment of the present invention;

FIGS. 9 to 16 inclusive are depictions of steps involved in a method of performing hip replacement surgery according to the preferred embodiment of the present invention, with FIGS. 15 and 16 being schematic depictions;

FIG. 17 is a depiction of a femur;

FIG. 18 is a plan view of another spacer according to the present invention;

FIG. 19 is a side view of the spacer shown in FIG. 18;

FIG. 20 is a front side view of an alternate spacer according to the present invention;

FIG. 21 is a top view of the spacer of FIG. 20;

FIG. 22 is a left side view of the spacer of FIG. 20;

FIG. 23 is a front side view of yet another alternate spacer according to the invention;

FIG. 24 is an enlarged partial view of a spacer showing the manner in which it engages an acetabular cup;

FIGS. 25, 26 and 27, are side, plan and end views respectively of another preferred embodiment of a jig according to the present invention;

FIG. 28 is a plan view of a drill;

FIG. 29 is a plan view of a scoop;

FIG. 30 is a plan view of an extractor;

FIGS. 31 and 32 are plan and side views respectively of a punch FIGS. 33 and 34 are plan views of respective left and right cup holders;

FIG. 35 is a plan view of a handle assembly;

FIG. 36 is a plan view of an alignment handle;

FIGS. 37 and 38 are side and plan views respectively of a first spacer;

FIGS. 39 and 40 are side and plan views respectively of a second spacer;

FIGS. 41 and 42 are side and plan views respectively of a third spacer;

FIGS. 43 and 44 are side and plan views respectively of an alignment frame;

FIG. 45 is a cross sectional view of a punch assembly;

FIGS. 46 and 47 are plan and end views respectively of fastener driver;

FIGS. 48 and 49 are plan and end views respectively of the drive head of the fastener driver shown in FIGS. 46 and 47;

FIGS. 50 and 51 are plan views of spacer members for operating on a left and a right hip respectively;

FIGS. 52 and 53 are plan and end views respectively of a medium width additional spacer;

FIGS. 54 and 55 are plan and end views respectively of a thick width additional spacer;

FIG. 56 is a side view of a pin extractor;

FIGS. 57 and 58 are plan and end views respectively of a pin;

FIGS. 59 to 62 are plan views of screws;

FIGS. 63, 64 and 65 are side, plan and end views respectively of another preferred embodiment of a jig according to the present invention;

FIG. 66 is a perspective view of a patient immediately prior to the commencement of hip replacement surgery, showing a pelvic holder and the site of the initial incision relative to the prominence of the greater trochanter;

FIG. 67 is a perspective view showing a jig according to a preferred embodiment of the invention being applied to a femur prior to osteotomisation of the femoral neck;

FIG. 68 is a perspective view showing an end of the extractor from FIG. 30 being applied to an osteotomisatised femoral head;

FIG. 69 is a perspective view showing part of a cup alignment tool being applied to the acetabulum of a patient; and

FIG. 70 is a perspective view showing part of a spacer alignment tool being applied to a patient's acetabulum.

### DETAILED DESCRIPTIONS OF PREFERRED EMBODIMENTS

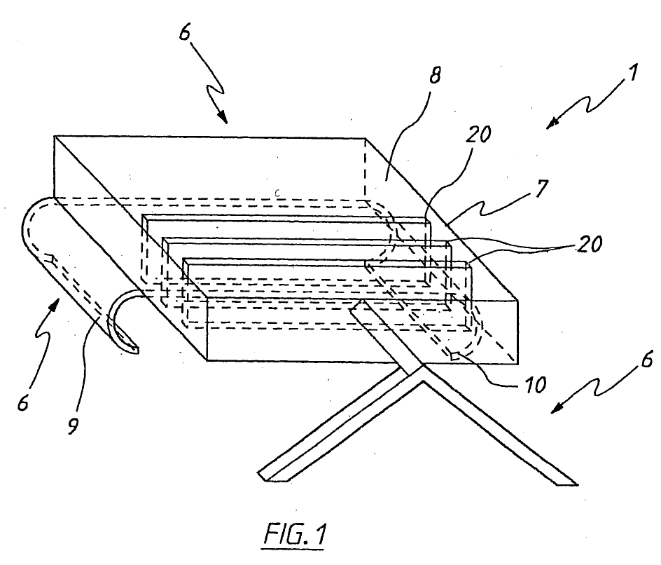

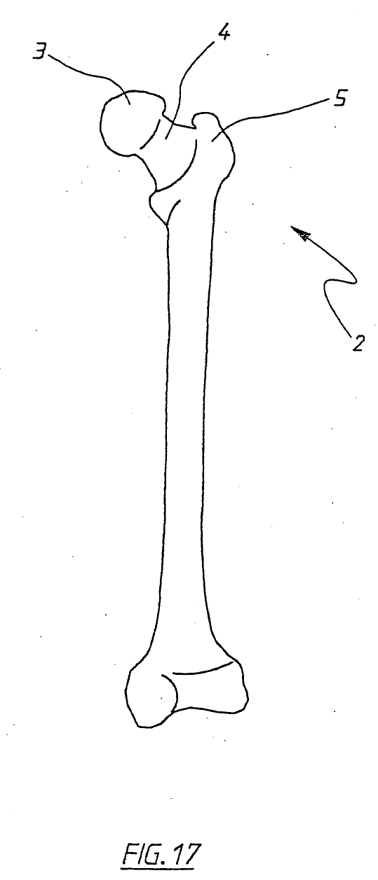

Referring to FIG. 1, the surgical jig 1 is adapted for application to a bone, for example a femur 2, however it will be appreciated that with detail changes the inventive concept may be applied to jigs adapted for application to other bones. As best shown in FIG. 17, the femur 2 includes a femoral head 3, a femoral neck 4 which is often, although not exclusively, the site of the cut, and the greater trochanter 5. If the surgeon chooses to use LINK T.O.P. acetabular cups and C.F.P. stems during hip replacement surgery, the femoral head 3 must be osteotomised (in other words, “cut off”), with the cut taking place along a line transverse to the femoral neck at a position approximately 1.5 cm from the base of the greater trochanter 5. Of course, other prosthetics, such as MARGRON THR and SP 2 equipment, may require the cut to take place at other sites on the femur 2, for example across the greater trochanter 5. For such applications the geometry of the jig 1 is adapted as required.

The jig 1 includes jig location means 6 adapted to mate with bone formations 3, 4 and 5 so as to locate the jig 1 in a predefined relationship with the bone 2. The jig 1 further includes surgical tool guide means 7 disposed relative to the jig location means 6 such that, when the jig 1 is mated with the bone formations 3, 4 and 5, the surgical tool guide means is adapted to guide a surgical tool, for example a reciprocating blade, towards a predefined site on said bone formation.

The jig 1 includes a body 8. One aspect of the jig location means 6 takes the form of first and second opposed lugs 9 and 10 disposed on the body 8. The lugs 9 and 10 are adapted to mate with the femoral neck 4. Each of the lugs 9 and 10 have an elongate dimension oriented such that, when the jig 1 is mated with the femoral neck 4, the elongate dimension of the lugs 9 and 10 is oriented in substantially the same direction as an elongate dimension of the femoral neck 4. This is best shown in FIG. 5 in which the lines 11 and 12 show the sites at which lugs 9 and 10 respectively engage the femoral neck. The lugs 9 and 10 are snap fittingly engageable with the femoral neck 4. Each of the lugs 9 and 10 are arcuate with a centre of curvature extending along the elongate dimension.

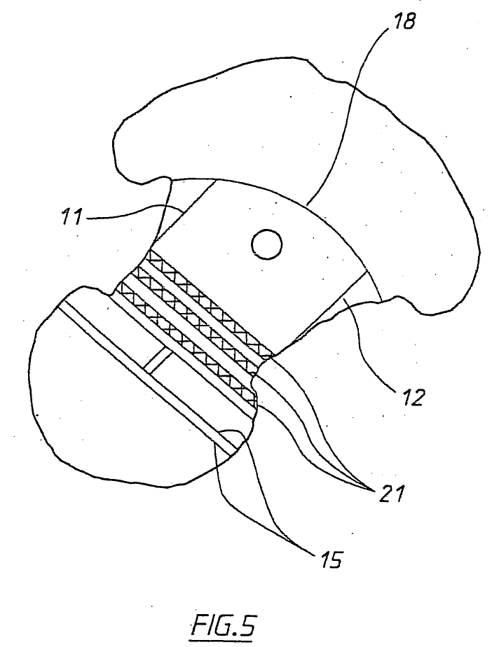

Another aspect of the jig location means 6 is a surface 13 adapted to receive a bone formation, for example a femoral head 3. In the preferred embodiment the surface 13 defines an inverted V shape, as best shown in FIG. 4. The inverted V shaped surface 13 is provided by an inverted V shaped member 14 disposed on the body 8 via a laterally extending member 16. The inverted V shaped member 14 is laterally disposed from the lugs 9 and 10. The section of the femoral head 3 over which the inverted V shaped member 14 is applied is shown by lines 15 in FIG. 5.

Yet another aspect of the jig location means 6 is a side wall 17 of the body 8 shaped so as to mate with a bone formation. More particularly, the side wall 17 is arcuate with a curvature shaped so as to abut a natural curvature of the greater trochanter 5. The line along which the side wall 17 substantially abuts the natural curvature of the greater trochanter 5 is best depicted by line 18 on FIG. 5.

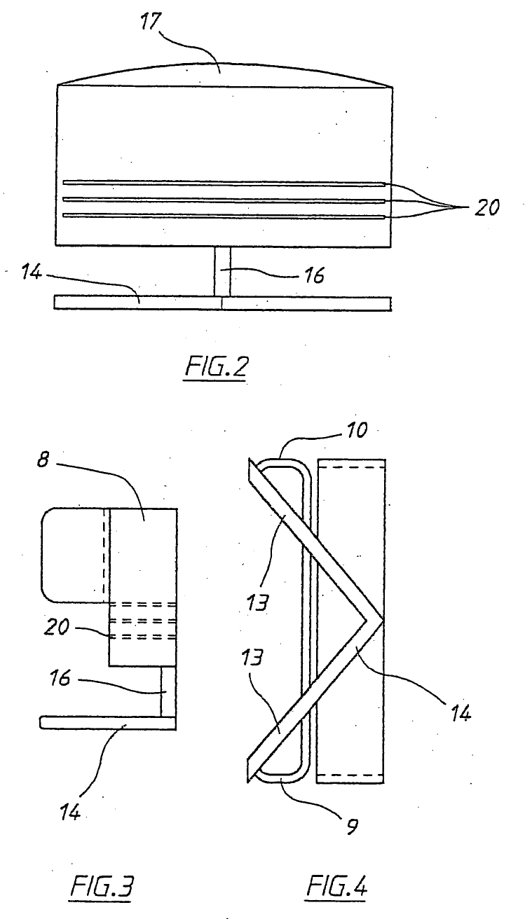

Hence the preferred embodiment of the jig 1 shown in FIGS. 1 to 5 mates with bone formations on the femur 2 via:
the lugs 9 and 10 hugging the femoral neck;
the V shaped member wrapping around at least part of the femoral head and
the arcuate side wall 17 abutting the natural curvature of the greater trochanter.

These location means combine to ensure that the jig 1 can be accurately placed onto the femur 2 and maintained in position to assist with aligning the osteotomisation of the femoral neck 4. Alternatively, the jig 1 may be oriented the other way around, with the inverted V shaped member 14 against the femoral neck 4 and the lugs 9 and 10 against the femoral head. This maybe further secured by driving one or more fasteners such as pins through body 8 or lugs 9 and 10 into the femoral head 3. Of course, damage to the femoral head 3 resulting from the fasteners is of little concern since the head 3 is to be osteotomised. The pinning of the jig 1 to the femoral head 3 can then be used to assist in removing the femoral head after the cut has been made, as discussed in more detail below.

The surgical tool guide means 7 is disposed intermediate the inverted V shaped member 14 and lugs 9 and 10. This assists in guarding against rotation or displacement of the jig 1 due to forces that may be transferred from the surgical tool to the jig 1. The surgical tool guide means 7 takes the form of one or more slots 20 disposed within the body 8. The preferred embodiment has three slots 20. Each slot is disposed at a different lateral position on the body 8 so as to allow the slots 20 to guide a surgical tool to more than one predefined site on the bone formation. The predefined sites are selected so as to correspond with different sized prosthetics. For example, one type of prosthetic requires the femoral neck 4 to be osteotomised at a position 1.5 cm from the base of the greater trochanter 5 and hence one of the slots 20 is disposed so as to guide the surgical tool toward this position. Each of the slots 20 extend transversely across the body 8 and are adapted to receive and guide a surgical tool, for example a reciprocating blade such as a jig saw. The positioning of the slots 20 once the jig 1 has been mated to the femur 2 is shown by lines 21 on FIG. 5. Other preferred embodiments (not illustrated) are adapted to guide different tools, for example one tool guide means is a hollow cylindrical member adapted to guide a rotating drill bit.

Although not shown in FIGS. 1 to 4, the preferred embodiment includes a handle disposed on said body 8. The handle has a length sufficient to extend externally of a patient when the jig 1 is mated to the bone formation. This allows the surgeon to manipulate the jig 1 past the flesh of the patient to mate with the femur 2.

The preferred surgical method according to the present invention is particularly suited to hip replacement surgery in the cases of osteoartritis, rheumatoid arthritis and osteonecrosis. However it is generally not recommended in the following circumstances:
for use on patients having gross anatomical deformity of the femoral head or neck; or
if the patient is obese, making the palpation of anatomical landmarks difficult.

During the preferred method of performing hip replacement surgery on a patient a postero-lateral surgical approach is preferred, although other surgical approaches will also suffice. Indeed, other surgical approaches may be necessitated if the postero-lateral surgical approach is contra-indicated, for example due to previous severe sciatic nerve injury.

The preferred method commences with step a)—exposing a greater trochanter 5, and femoral neck 4 of the patient. This entails making an initial incision in a thigh of the patient of between 3 cm and 7 cm, preferably 5 cm, in length. One end of the incision commences approximately 1 cm to 3 cm behind the greater trochanter 5 and curves backwards therefrom. The posterial aspect of the femoral neck 4 is exposed by detaching a tricipital tendon of the patient, which may be tagged for later re-attachment. A capsulectomy is also performed to fully expose the femoral neck 4. It is important to note that the femoral head 3 is not dislocated at this stage. The leg of the patient is maintained in a position approximately 45° from vertical, preferably by a leg holder. It may also be of assistance for the surgeon to make use a head light.

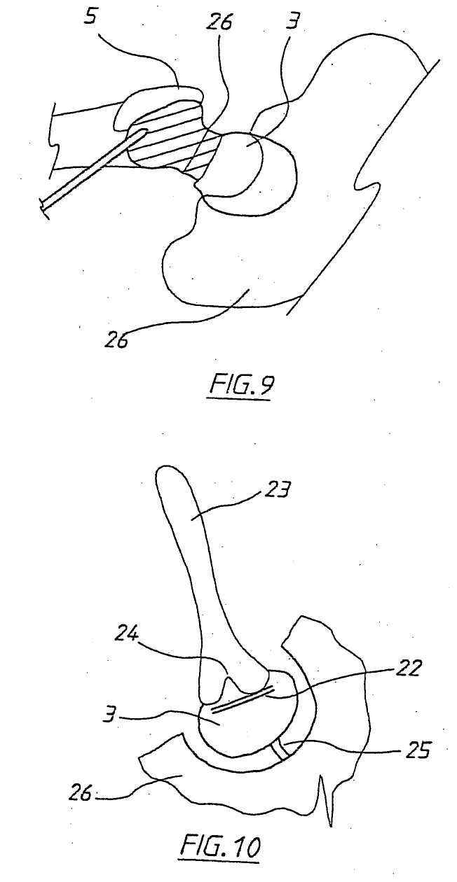

Step b) involves applying a jig 1 having surgical tool guide means 7 to the femur 2 such that the surgical tool guide means 7 is proximal the femoral neck 4, as shown in FIG. 9. The jig 1 is preferably as described above. Properly mating the jig 1 to the femur 2 results in the surgical tool guide means 7 being disposed approximately at a predefined distance from the base of the greater trochanter 5. More particularly, the predefined distance is between 10 mm and 20 mm, and in the preferred embodiment, at least one of the slots 20 is disposed 15 mm from the base of the greater trochanter 5.

Optionally, the surgeon may form a notch 22 in the femoral neck 4 on the femoral head 3 side of the osteomological cut site 26. The notch 22 is preferably formed prior to the osteomological cut. The notch may be formed by briefly impacting a reciprocating jig saw blade against the femoral neck 4 or head 3.

Step c) involves using the surgical tool guide means to guide a cutting tool so as to osteotomise the femoral neck. Typically the blade of a jig saw is fed through one of the slots 20 so as to rest against the femoral neck 4 at the osteotomological site, although other cutting means may be employed. The jig saw is then turned on causing the cutting blade to reciprocate and the jig saw blade is progressively moved transverse to the femoral neck 4 as guided by the slot 20 until the femoral head 3, and part of the femoral neck 4 are osteotomised.

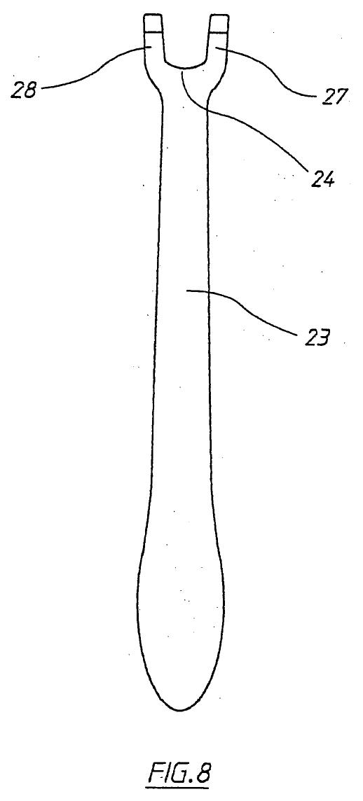

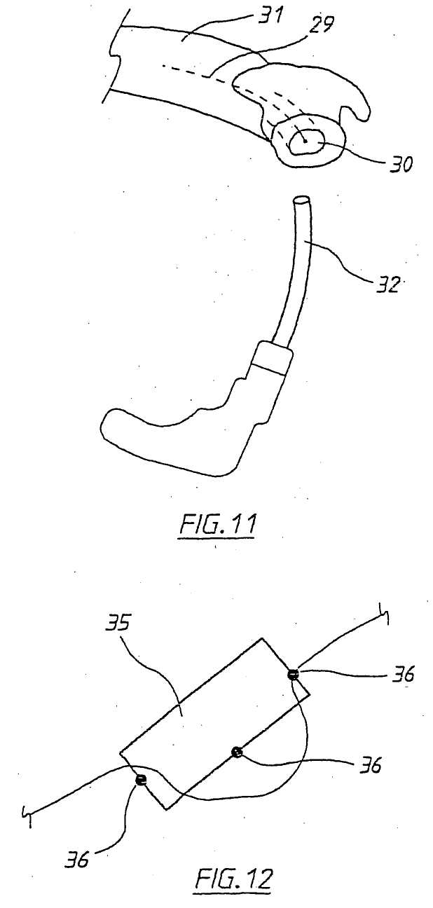

Step d) entails removing a severed femoral head from the patient's hip joint. A number of different techniques may be employed to achieve this. For example a scoop 23 as shown in FIG. 8 may be used to lever the severed femoral head 4 out of the hip joint. The notch 22 may provide a convenient site from which to lever the femoral head 3 with the scoop 23, as shown in FIG. 10. A portion 24 of the scoop 23 is provided with cutting means, such as a sharpened edge, to assist in severing the tendon 25 which retains the femoral head 3 inside the hip 26. The sharpened edge 24 is disposed intermediate two projections 27 and 28 which assist the surgeon to manipulate the scoop 23 so that the sharpened edge 24 is directed toward the tendon 25. In other words, the surgeon places the scoop 23 so that the projections 27 and 28 are on either side of the tendon 25, then the scoop is displaced along its longitudinal axis towards the tendon 25 to allow the sharpened edge 24 to sever the tendon 25.

An alternative method for removing the femoral head 4 may be employed if the jig has been fixed by pins to the femoral head 3 as mentioned above. In this case force may be applied to the jig 1 which is transferred through the pins to the femoral head 3. In some cases this force alone may suffice to remove the femoral head 3 from the hip joint. In other cases this force may assist other removal means, such as the scoop 23 mentioned above.

It will be appreciated by those skilled in the art that osteotomising the femoral neck 4 prior to removing the severed femoral head 3 advantageously avoids the prior art requirement to firstly dislocate the hip joint, thereby avoiding or minimising the damage this can inflict, and simplifying the surgical procedure. Following removal of the severed femoral head 3, the hip replacement surgery may be completed in accordance with prior art techniques. However a number of further improvements to the known surgical procedure are outlined below.

A prior art method for implanting a prosthetic stem into the femoral shaft involves driving a bone compressor into the femoral canal. An alternative provided by the preferred embodiment of the present invention includes the steps of:
e) placing a guide wire 29 into the femoral canal 30 of a femoral shaft 31 of the patient; (refer to FIG. 11)
f) using the guide wire 29 to guide a flexible reamer 32 so as to bore a aperture into the femoral canal 30; and
g) inserting a prosthetic stem 33 into the aperture such that a first end 34 of the stem 33 protrudes from an end of the femoral shaft 31. Alternatively, it is possible to use a removable trial stem in the place of the final stem 33. Then, once the replacement hip joint has been checked and a satisfactory result obtained, the true components are implanted in the place of the removable trial components.

The prior art method for reaming a cavity into the acetabulum involves aligning the reaming tool by eye. In contrast, the step of reaming the acetabulum to accept a prosthetic acetabular cup according to the preferred embodiment of the present invention includes the steps of:
h) placing the first end 34 into an acetabulum of the patient;
i) inserting spacers 35 (also referred to as lollipops) intermediate the end of the femoral shaft 31 and the acetabulum;
j) using a spacer 35 to determine reference points 36 on an acetabulum of the patient (refer to FIG. 12); and
k) using said reference points 36 to provide guidance for reaming of the acetabulum.

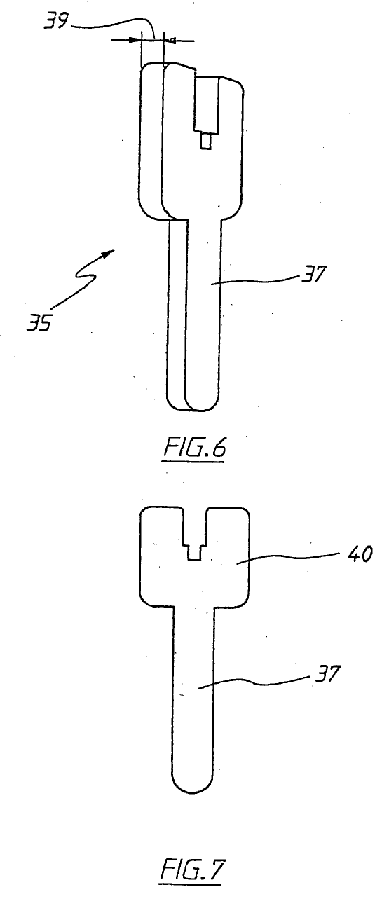

More particularly, examples of the spacers 35 used in step i) are as shown in FIGS. 6 and 7. Their purpose is to place the femur approximately in the position it will assume once the operation has been completed. Markings 40 on the spacers 35 provide guidance for determining the reference points in step j). Preferably the reference points 36 are burnt onto the acetabulum using a heating element similar in function to the heated tip of a soldering iron. The spacers 35 include a handle 37 and a slot 38 for placement onto the first end 34 of the prosthetic stem 33.

The center of the reference points 36 established in step j) provides the starting point for reaming of the acetabulum in step k). This provides a more accurate and consistent alternative to the prior art reaming technique whereby the alignment is performed by eye.

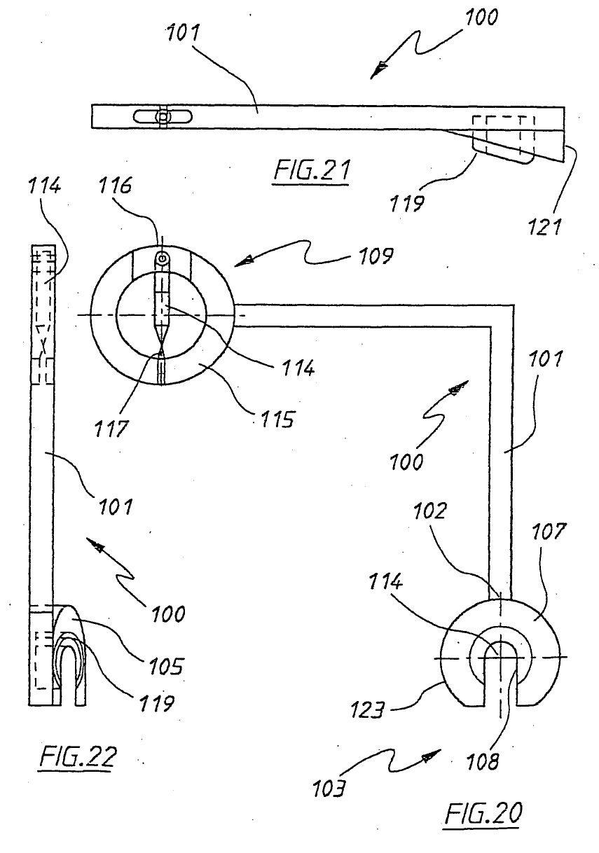

Other example of spacers 100 which may be employed in steps i) and j) are shown in FIGS. 20 to 24. These spacers 100 include an outer circumferential edge 123 having a radius of curvature substantially corresponding to a radius of curvature of a prosthetic acetabular cup 104. In the preferred embodiment the radius of curvature of the outer circumferential edge 123 matches a radius of curvature of the smallest of a range of prosthetic acetabular cups 104. At step j) the surgeon uses the edge 123 as a guide when burning reference points 36 onto the acetabulum prior to the reaming of step k).

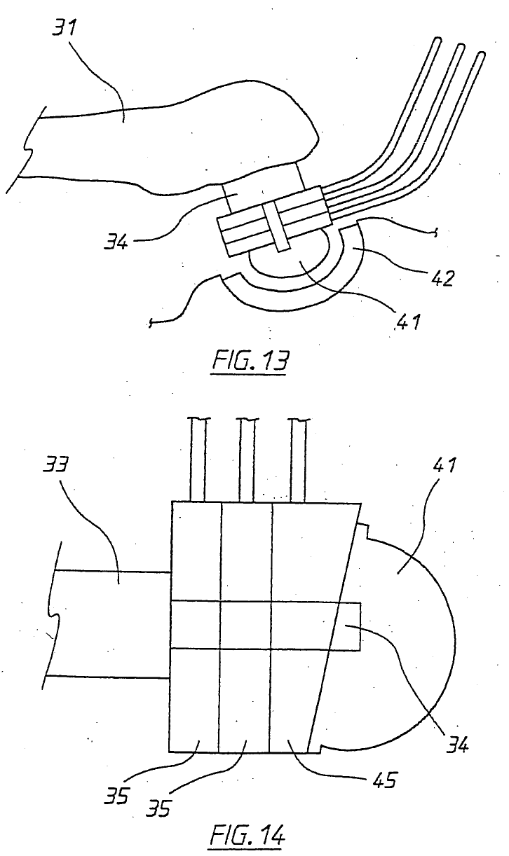

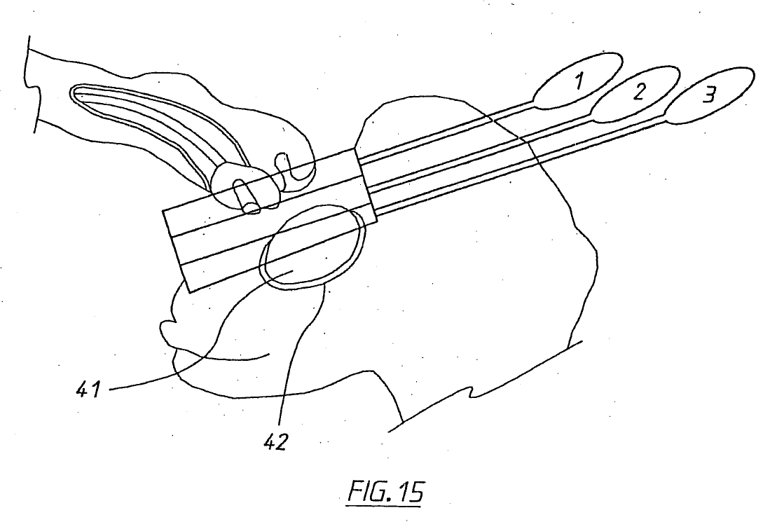

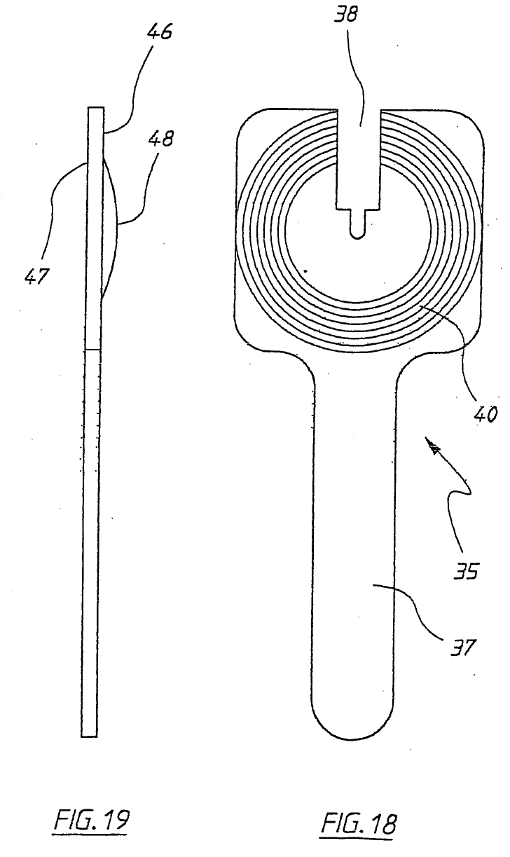

The prior art hip replacement surgical method involves the surgeon making an educated estimate as to the required size of prosthetic components, such as prosthetic neck/head length and acetabular cup size. Then the hip joint is temporarily assembled using trial components having the estimated size. If the resultant trial joint is not satisfactory, for example due to incorrect tissue tension, the joint is disassembled and differently sized trial components used. The preferred method of the present invention provides an alternative to this prior art procedure by once again utilising the spacers 35. As best shown in FIG. 18, one or more of the spacers 35 or 45 has a centrally disposed spherical surface 48 which mimics the shape of a femoral head so as to function as a trial femoral head. In some embodiments the spherical surface 48 is a half hemisphere which corresponds to part of a prosthetic head. The spacer 35 or 45 which has the spherical surface 48 is disposed on the first end 34 of the stem 33 closest to the acetabular cup so that the spherical surface 48 faces the acetabular cup. The thickness 39 of each spacer 35 is selected with regard to available sizes of prosthetic components. For example, one prior art prosthetic component is available in three sizes, 40 mm, 43.5 mm and 47 mm. In this case the thickness 39 of at least two spacers 35 is 3.5 mm, so as to correspond to the size increments between available prosthetic components. These two spacers have flat parallel faces 46 and 47. The effects of utilising differently sized prosthetic components may be mimicked by utilising varying numbers, or thicknesses, of spacers 41. For example, the edges of three spacers 35 are shown applied to the first end 34 of the prosthetic stem 33 in FIG. 14. This mimics the spacing achieved through use of the 47 mm prosthetic component. If two of the spacers 35 are utilised, this mimics the spacing associated with a 43.5 mm prosthetic component, and so forth. As best shown in FIG. 14, another spacer 45 is wedge shaped. The wedge shape determines the anteversion (or “orientation”) of the prosthetic acetabular cup and the prosthetic femoral stem. A converse wedge shape is required if an anterior or antero lateral surgical approach is adopted. The wedge shape tapers from a maximum width of 14 mm to a minimum width of approximately 3.5 mm.

Other embodiments of spacers 100 adapted to control the anteversion of the prosthetic cetabular cup 103 are shown in FIGS. 20 to 24. The spacer 100 includes a handle 101 defining a first end 102. Alignment means 103 are disposed at, or adjacent to, the first end 102. The alignment means 103 is adapted to mate with a prosthetic acetabular cup 104 such that, in use, the cup 103 is positioned in a predefined relationship with reference to an acetabulum of a patient.

The alignment means 103 defines a plane 105 adapted to abut a corresponding plane 106 of the prosthetic actabular cup 104. The alignment means 103 includes engagement means taking the form of one or more lugs 119 adapted to engage an internal concave surface 120 of the prosthetic acetabular cup 104. The plane 105 is defined by a semi circular surface 107 and is disposed at an acute angle 111 with reference to a plane 112 defined by the handle 101. Hence, the alignment means 103 takes the form of a wedge shaped member 110 disposed at the first end 102. Preferably the angle 111 is within the range of 5° to 25°. More preferably the angle 111 is within the range of 10° to 20° and in the preferred embodiment the angle 111 is approximately 15°. This angle 111 is chosen so as to correspond with a natural angle of inclination of the hip joint.

In the preferred embodiment the geometry of the alignment means with reference to the handle is chosen to be appropriate for a postero-lateral surgical approach to hip surgery. Of course, other surgical approaches may necessitate slightly varied geometries, for example the different handle geometry shown in 23 as compared to FIG. 20 is suited to other surgical approaches. The general goal is to ensure that when the spacer 100 is applied to an exposed end 34 of a prosthetic femoral stem 33, the alignment means 103 positions the prosthetic acetabular cup 104 with an anteversion substantially identical to the desired anteversion of the prosthetic cup 104 upon completion of the surgery.

The alignment means 103 includes a slot 108 which is adapted to receive a protruding end 34 of a prosthetic femoral stem 33. Once the end 34 of the stem 33 is placed inside the slot 108, the spacer 100 engages the stem 33 in a similar manner to the way in which spacer 45 engages the stem end 34 in FIG. 14.

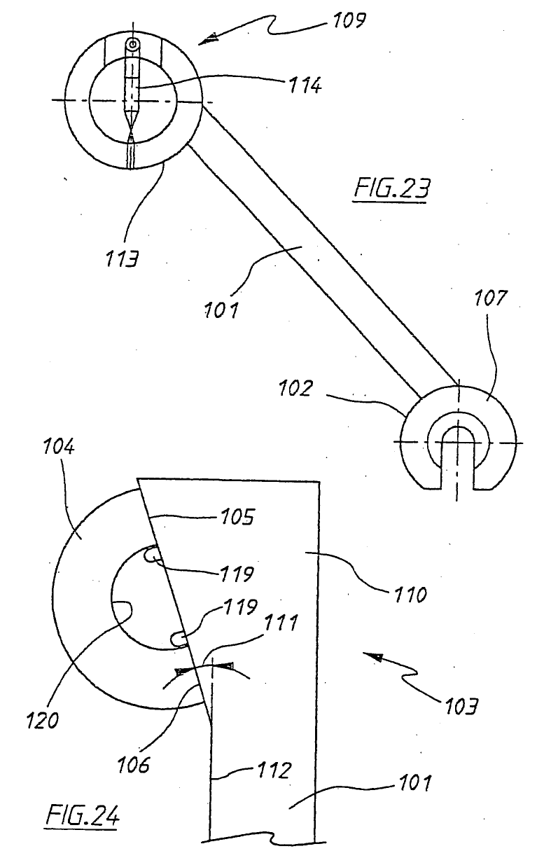

The angular inclination of the prosthetic acetabular cup relative to the prosthetic stem 33 should mirror the natural geometry of the hip joint as accurately as possible. As the spacer 100 is rotated about an axis of rotation 118, the direction of the 15° angular inclination changes. To provide the surgeon with guidance as to whether the alignment means 103 is positioned in said predefined relationship with reference to an acetabulum of a patient, an orientation guidance means 109 is disposed upon the spacer 100 at or adjacent to second end 113. The orientation guidance means 109 includes a plumb bob 114 rotatably disposed upon the spacer 100 so as to hang substantially vertically under the influence of gravity. The plumb bob 114 is rotatably affixed to a circular member 115 of the spacer 100 by a pin 116. A reference point 117 is disposed on said spacer 100 such that the plumb bob 114 hangs in alignment with the reference point 117 when the alignment means 103 is positioned in said predefined relationship with reference to an acetabulum of a patient. In other words, when the spacer 100 has assumed the required rotational position with regard to axis of rotation 118, the plumb bob 114 hangs in alignment with the reference point 117, as shown in FIGS. 20 and 23. Hence, if the patient is lying on its side, the slot 108 is mated with the end 34 of the prosthetic shaft, and the spacer 100 is rotated about axis 114 until the plumb bob 114 is aligned with the reference point 117, then the prosthetic acetabular cup 103 will have the required anteversion in relation to the stem 33. That is, when correctly used, the spacer ensures that the thicker end 121 of the wedge shaped member 110 is disposed towards the front of the patient and the thinnest end 122 is disposed towards the rear of the patient. Hence, the 15° inclination is directed towards the outer front side of the patient, mirroring the natural geometry of the hip joint. More particularly, the preferred method of determining the required size of prosthetic component includes the steps of:
l) placing a trial head 41 onto the first end 34;
m) placing a trial acetabular cup 42 into the reamed actabulum (refer to FIG. 13);
n) placing said trial head 41 into said trial acetabular cup 42; and
o) inserting one or more spacers 35 intermediate the end of the femoral shaft 31 and the trial acetabular head 41 until a required fit between the trial head 41 and the trial acetabular cup 42 is obtained (refer to FIGS. 13, 14, 15 and 16); and
p) correlating the number or size of spacers 35 with a required size of prosthetic component.

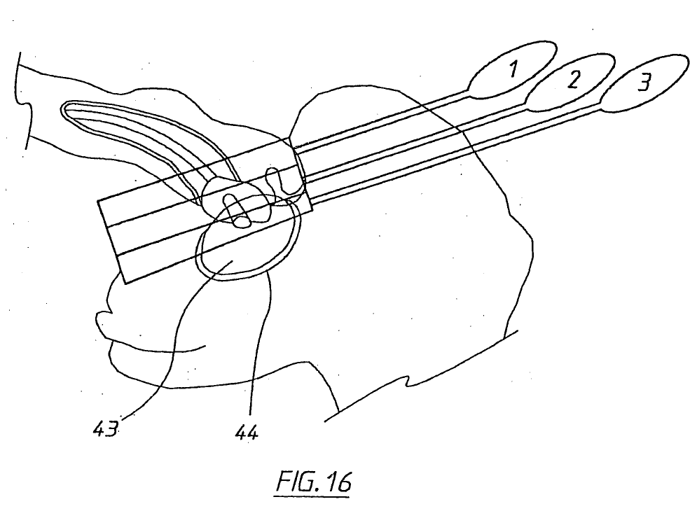

The surgeon determines in step o) whether a required fit between the trial head 41 and the trial acetabular cup 42 has been obtained by examining factors such as the tissue tension, component alignment, leg length, etc. Once the required size of the prosthetic component has been determined in step n) the trial prosthetic components 41 and 42 (FIG. 15) are replaced by the final prosthetic components 43 and 44 (FIG. 16), although the acetabular cup is not impacted at this point. A further use of the spacers 35 determines the final and accurate orientation of cup 44 to stem 33, then cup 44 is impacted firmly to permanently set it into place. The liner is put into the acetabular cup 44, followed by the prosthetic head 43 which is reduced. Once the range of motion of the new joint has been confirmed, the layers are closed in and the tricipital tendon re-attached.

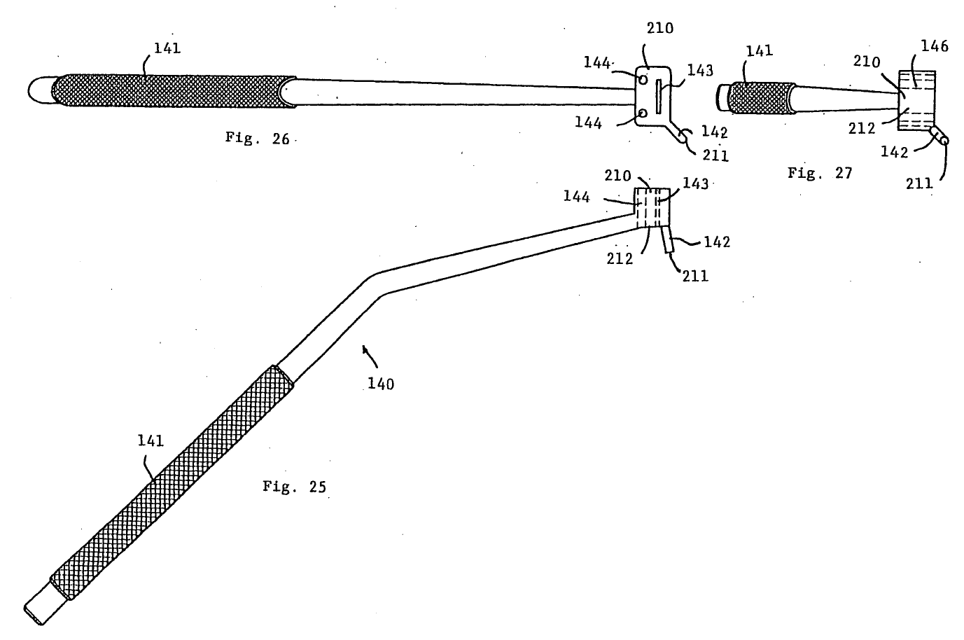

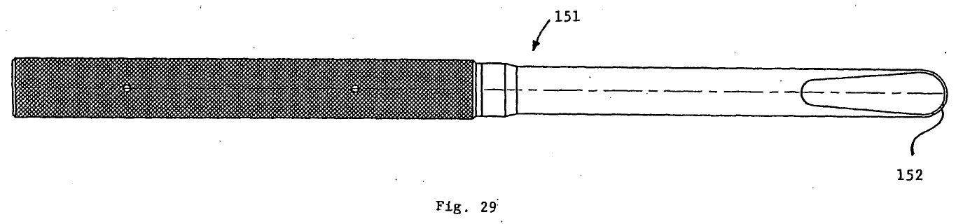

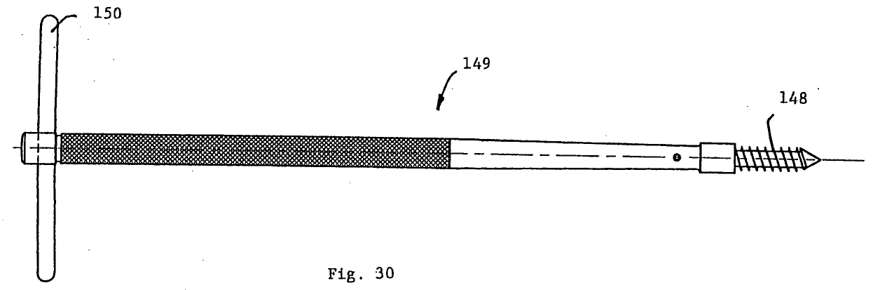

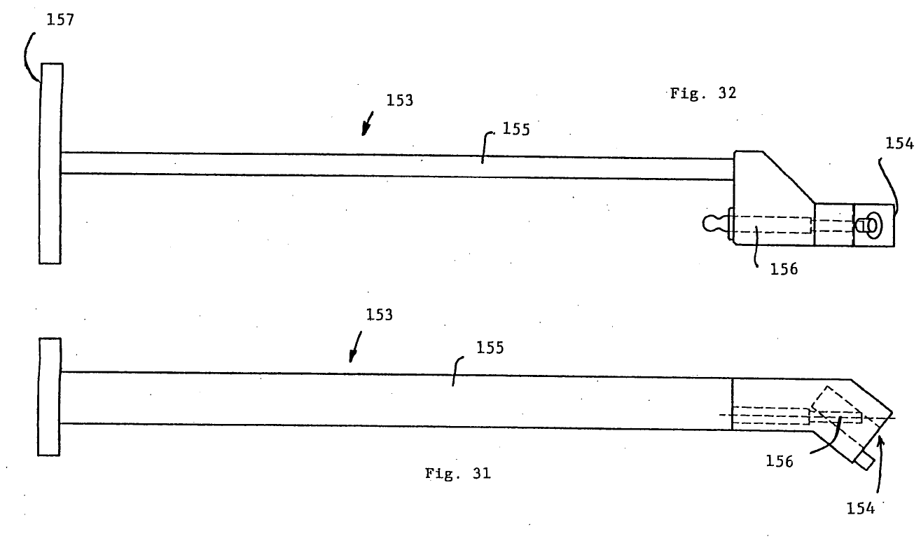

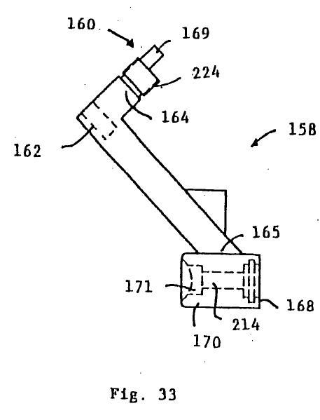

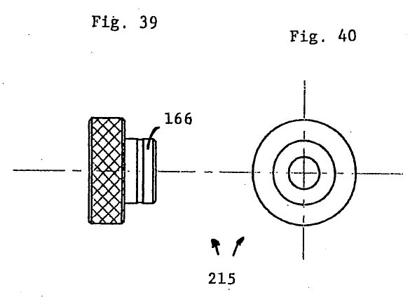

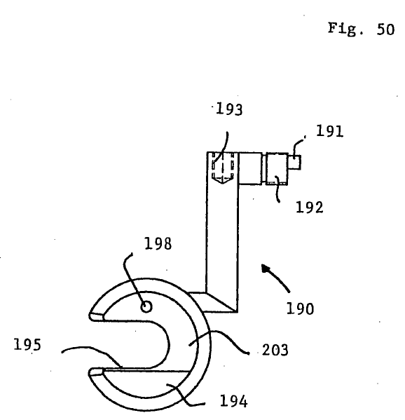

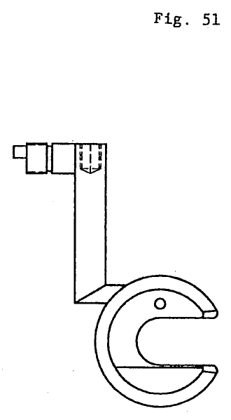

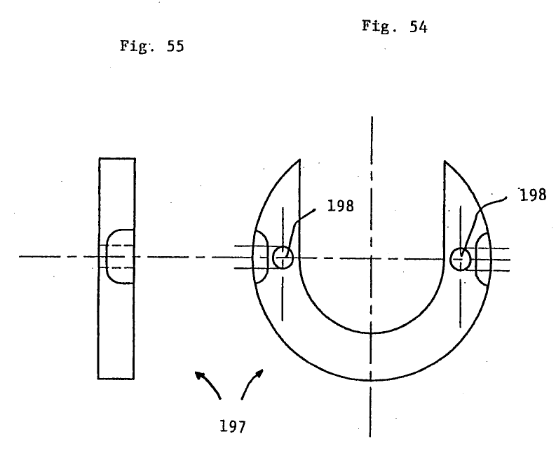

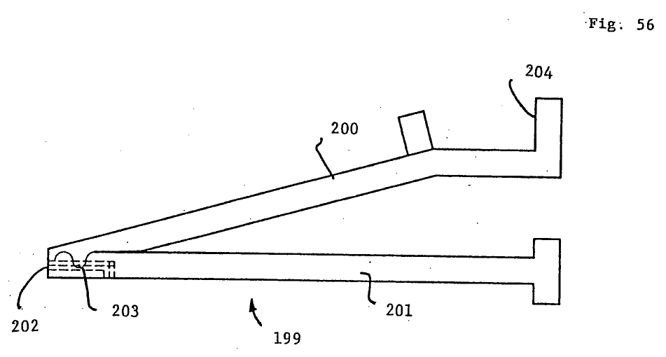

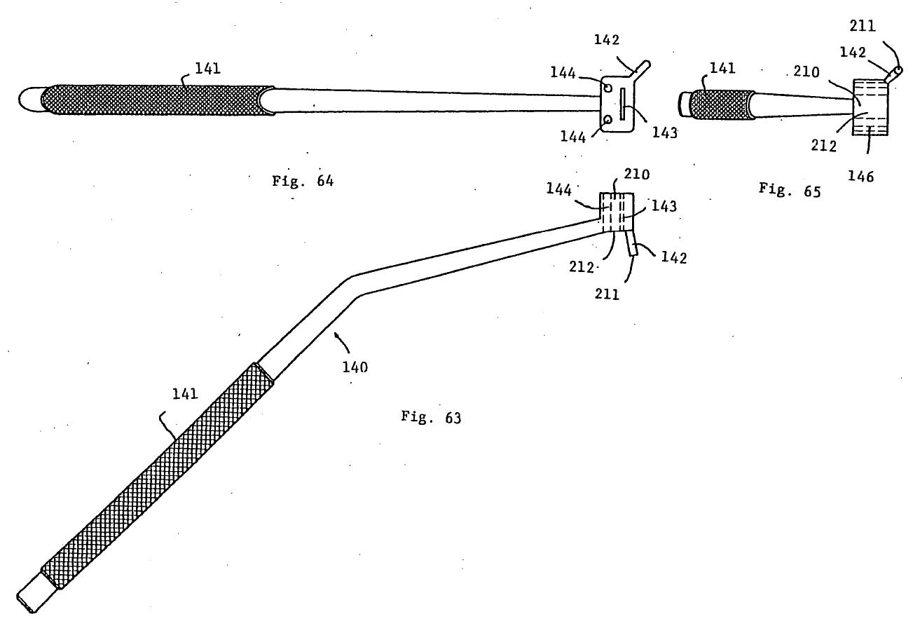

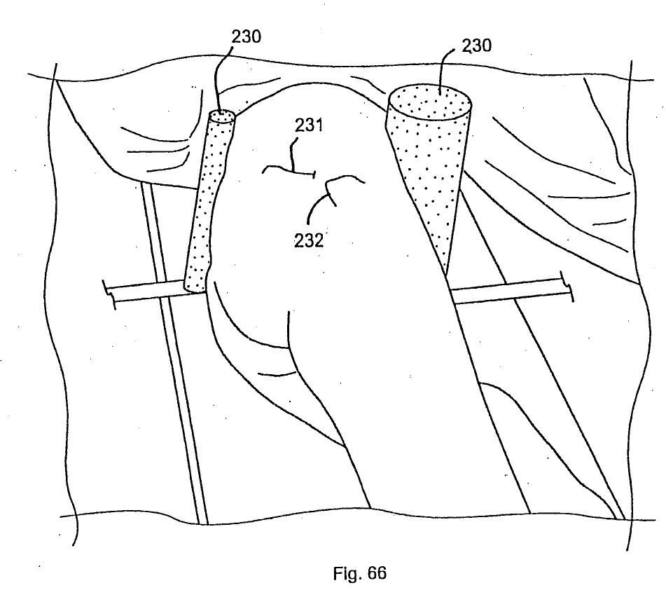

The instruments illustrated in FIGS. 25 to 65 collectively form a preferred embodiment of a kit for performing the preferred surgical procedure. This preferred surgical procedure commences with positioning the patient in the lateral position with the operative side uppermost as best shown in FIG. 66. Preferably a pelvic holder 230 is employed to maintain the patient in the required position. Also preferably the patient's limb temperature is lowered prior to and during surgery.

Exposure of the operative site commences with an approximately 5 cm long incision along a line 231 which is made approximately 2 cm behind the prominence (shown as line 232 in FIG. 66) of the greater trochanter 5. The tensor fasia is then incised and the surgeon performs a blunt dissection until the neck 4 of the femur 2 is exposed. Self-retainers are utilised below the skin line and intermittently. The posterior aspect of the femoral neck 4 is exposed by detaching the tricipital tendon and short external rotators which are tagged for later re-attachment. A capsulectomy exposes the trochanteric fossa (that is, the base of the greater trochanter 5) and further exposes the femoral neck 4 and then accessible acetabular osteophytes are removed. Unlike prior art hip replacement surgical methods, the head 3 is not removed at this point. The surgeon may choose to use a headlight to assist visibility. Preferably any irrigating fluid used during the surgery is chilled so as to minimise bleeding.

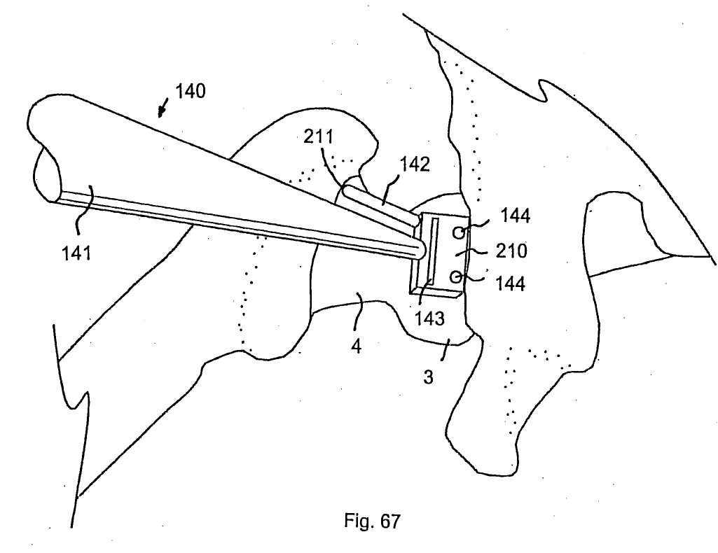

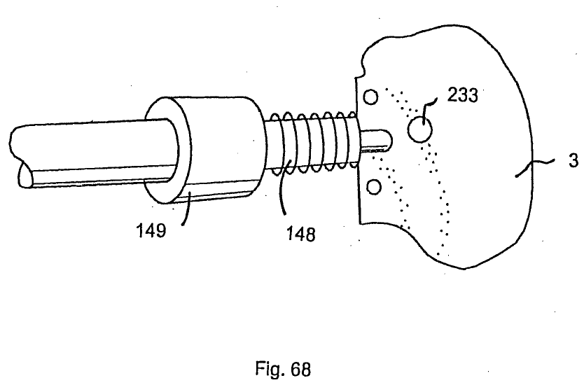

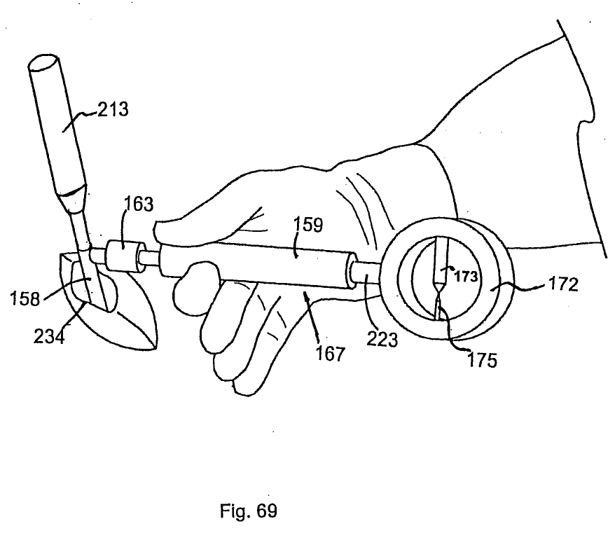

The leg is placed in a leg holder and maintained at approximately 45 degrees to the vertical. The surgeon grasps the jig 140 by its handle 141 and manipulates the head 210 of the jig through the patient wound and onto the femoral neck 4, as best illustrated in FIG. 67. The head 210 of the jig 140 includes jig location means in the form of an elongate rod which acts as a spacer 142. The spacer 142 has an end 211 which abuts the trochanteric fossa so as to position the slot 143 of the jig 140 at the required position, which, in the preferred embodiment, is between 5 mm and 25 mm, and most preferably 15 mm, from the trochantic fossa.

The first version of the jig 140, as shown in FIG. 67, is adapted for use on the patient's left hip joint. Another version of the jig 140, is adapted for use on the patient's right hip joint. The only substantive difference between the two versions is that the spacer 142 is disposed on opposite sides of the head 210.

Additional jig location means are provided by a surface 212 adapted to receive a bone formation. This surface 212 is provided by contours 146 on the base of the head 210 which are adapted to mate with contours of the femur.

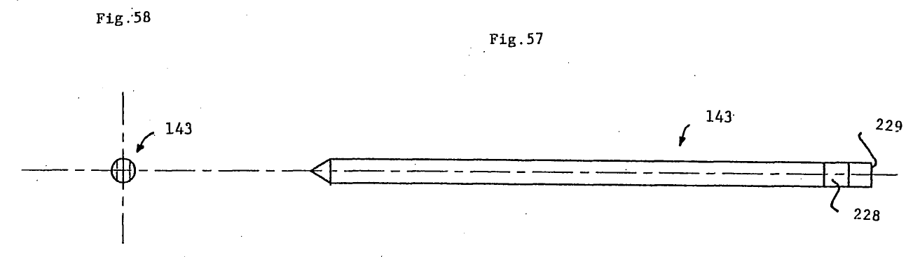

Once the jig has been correctly positioned, fixing means such as pins, 143, as shown in FIGS. 57 and 58, are driven through holes 144 in the head 210 of the jig 140 and into the femoral head 3.

The slot 143 is oriented at an acute angle to the elongate dimension of the rod 142, with the angle being dependent upon the required angle of cut to suit the particular prosthetic components being used in the surgery. The slot 143 functions as a surgical tool guide means which is positioned by the jig 140 at the correct position for osteotomisation of the neck 4. Advantageously, osteotomisation takes place whilst the femoral head 3 is still disposed within the acetabulum. The surgeon may also perform a secondary proximal wedge osteotomy if deemed necessary.

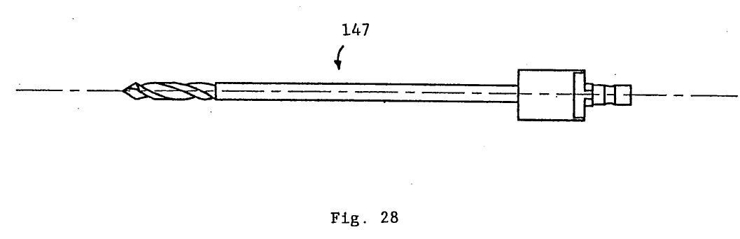

Subsequent to the osteotomisation of the neck 4, the femoral head 3 is folded forward then removed using one or more of a number of possible options. The drill 147, as shown in FIG. 28, may be used to drill a hole 233 into the femoral head 3. The self-tapping threaded member 148 of the extractor 149 is then tapped into the hole 223 by screwing the handle 150. Once lodged into the femoral head 3, the surgeon exerts an outwardly directed force onto the handle 150 which is, in turn, exerted onto the femoral head 3 so as to remove the femoral head 3. Another option, which may or may not be used in conjunction with the extractor 149 is provided by the scoop 151. The distal end 152 tapers to an edge which can be wedged under the femoral head 3 to assist with levering the head 3 out of the acetabulum. The tapered end 152 can also be used to sever the tendon holding the head 3 within the acetabulum. Other removal options include sectioning, or dicing, the head 3.

For the next surgical step the leg of the patient is supported perpendicular to the operating table. The proximal femur is prepared in accordance with standard prior art techniques using a trocar awl and a guide. Pointed Hohmanns (that is, retractors) are placed on either side of the femoral neck 4. Alternatively a broad blunt Hohmann may be applied under the neck 4 so as to elevate the neck 4.

A bone compressor is mountable to the punch 153 at the driving surface 154 and is held in place by a pin 156. The bone compressor is driven into the longitudinal axis of the femur using the punch 153 by striking the proximal end 157 with a hammer, mallet or other suitable tool. This process forms a hollow in the femoral canal for subsequent installation of a stem 34 (see below). The driving surface 154 of the punch 153 is laterally displaced from the elongate longitudinal member 155 to allow the surgeon to more easily drive the bone compressor in the correct direction whilst negotiating and manipulating the punch 153 through the patient wound. This assists in minimising the risk that the bone compressor will be driven into the femur at an angle which may cause an outer edge of the femur to crack. A calcar reamer is then used to form a circular plane on the end of the neck 4. The femur is now ready for insertion of a prosthetic stem, which takes place after the insertion of the acetabular cup as described below.

The surgeon next positions the patient's limb in a neutral position on the operating table to prepare for reaming of the acetabulum. A pointed Hohmann is then placed over the anterior margin of the acetabulum. The acetabulum is then reamed in a stepwise manner using increasing diameters.

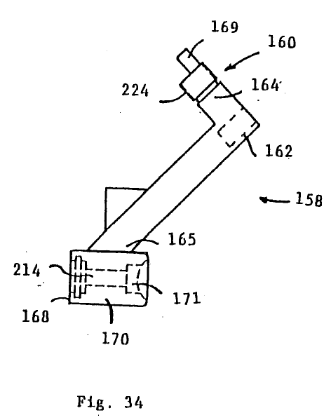

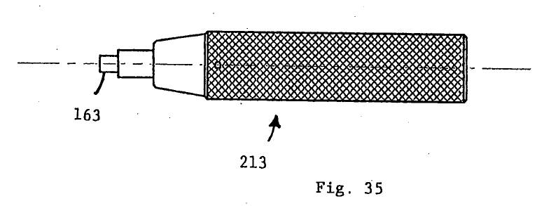

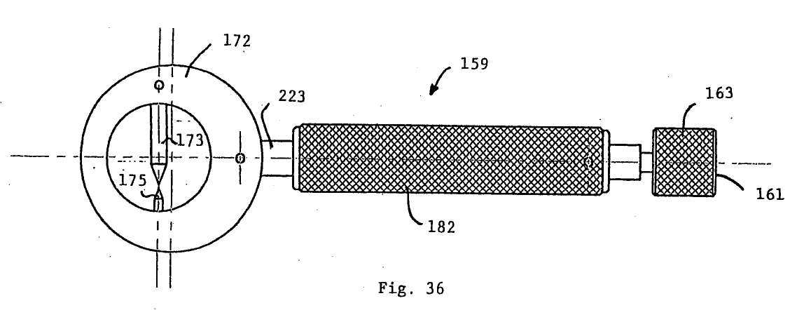

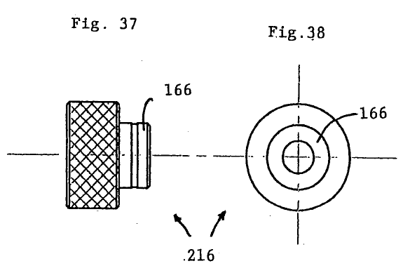

To insert the prosthetic acetabular cup into the reamed acetabulum, a cup alignment tool 167 is assembled from the following three components: the alignment handle 159 shown in FIG. 36, the handle 213 shown in FIG. 35 and either the left cup holder 158 or the right cup holder 158 as shown in FIGS. 33 and 34 respectively. The alignment handle 159 includes a body 182 which defines a distal end 161 and a proximal end 223, with a handle 182 intermediate the ends. A gauge, in the form of a plumb bob 172, is disposed at the proximal end 223. The gauge 172 is adapted to indicate whether the prosthetic acetabular cup is in a predefined orientation with respect to at least a first plane in a referencial system. A connector which includes an internally threaded collar 163, is disposed at the distal end 161.

Either the left or the right cup holder 158, as required, is attached to the alignment handle 159 via attachment means 160 disposed on a proximal end 164 of the cup holder 158. More particularly, the attachment means 160 includes a projection 169 which keys into the connector on the distal end 161 of the alignment handle 159 to ensure correct relative alignment between the two components. Once keyed into each other, the internally threaded collar 163 engages the external thread 224 on the cup holder 158 to secure the two components together.

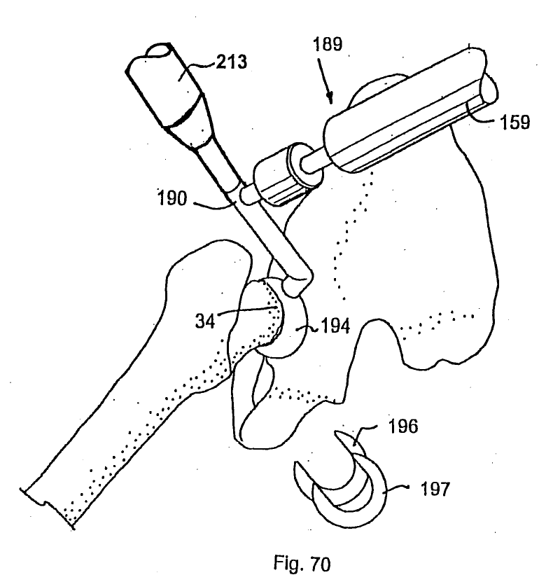

The handle 213 provides the surgeon with additional leverage when manipulating the cup alignment tool 167. To connect the handle 213 to the cup holder 158, an external thread 163 on the handle 213 threadedly engages an internal thread 162 disposed within the cup holder 158. The resulting cup alignment tool 167 is as illustrated in FIG. 69 (although with part of the cup holder 158 obscured due to insertion into a patient wound 234).

The next step is to attach a prosthetic acetabular cup of appropriate size onto the cup alignment tool 167. Depending upon the size of the prosthetic acetabular cup, a spacer 174, 215 or 216 may be required. Spacers of various widths, as illustrated in FIGS. 37 to 42, are engagable with a first end 168 of a boss 176 which is disposed on the distal end 165 of the cup holder 158. More particularly, a projection 166 disposed on the spacer 174 is press fittingly engagable with a corresponding aperture 214 provided in the first end 168 of the boss 170.

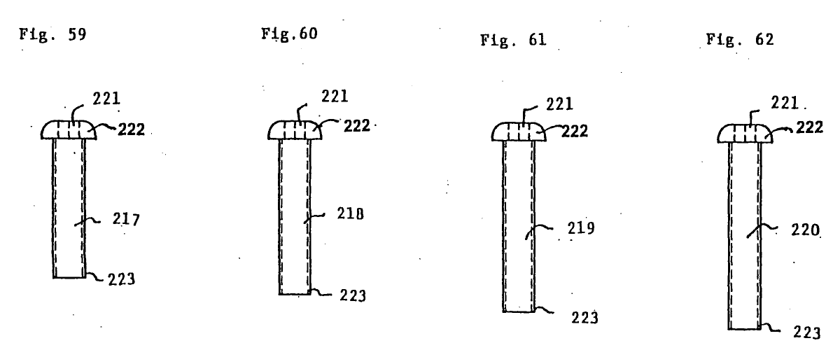

The surgeon selects a spacer 174, 215 or 216 having an appropriate width to match the size of the prosthetic cup being inserted into the patient's hip. For the smallest prosthetic acetabular cup no spacer 174 is required. The spacers 174, 215 and 216 are consumable items made from a plastics material which is designed to withstand only a single operative use. The kit of instruments includes four screws 217, 218, 219 and 220 with differing lengths as illustrated in FIGS. 59 to 62 respectively. Hence, this screw and spacer arrangement caters for four sizes of prosthetic acetabular cup respectively corresponding to:
prosthetic acetabular cup size 1: screw 217 and no spacer;
prosthetic acetabular cup size 2: screw 218 and spacer 174;
prosthetic acetabular cup size 3: screw 219 and spacer 215; and
prosthetic acetabular cup size 4: screw 220 and spacer 216.

Preferably the screws 217, 218, 219 and 220 feature an allen head socket 221. Each screw has a length which is just sufficient to extend axially through the aperture 214 of the boss 170, with the head 222 of the screw adjacent the second end 171 of the boss 170 and the threaded end 223 emerging just past the first end 168 of the boss 170 and also just past whatever spacer 174, 215 or 216 may be engaged with the first end 168. The emerging tip of the threaded end 223 is threadedly engagable with the prosthetic acetabular cup 104. In this way the prosthetic acetabular cup is mounted onto the cup alignment tool 167. More particularly, the acetabular cup is engaged with the cup holder 158 such that a concave face of the acetabular cup faces towards the second end 171 of the boss 170.

The surgeon then manipulates the cup alignment tool 167 into the wound 234 as shown in FIG. 69 such that the cup is adjacent the reamed acetabulum. It is now necessary to orient the cup alignment tool 167 to ensure that the prosthetic acetabular cup is in an anatomically correct orientation for insertion into the reamed acetabulum. This is achieved with reference to the gauge 172 on the alignment handle 159 in combination with an alignment frame 176.

The gauge 172 has a needle 173 which functions as a plumb bob. The needle 173 is rotatably disposed on the alignment handle 159 so as to hang substantially vertically under the influence of the local gravitational field. The gauge allows the surgeon to correctly align the acetabular cup in at least a first plane by positioning the cup alignment tool 167 such that the rotatable needle 173 of the plumb bob 172 is aligned with a reference point in the form of the fixed needle 175.

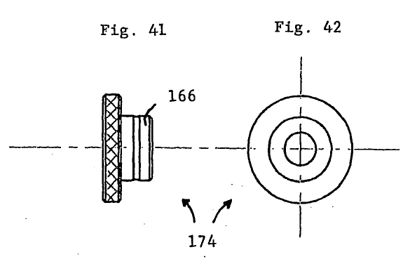

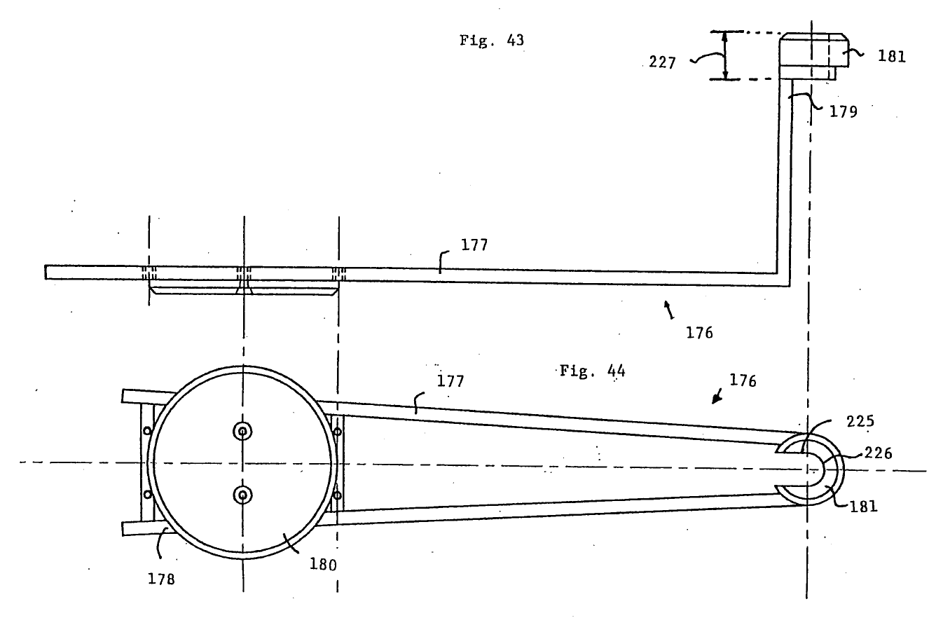

The alignment frame 176 shown in FIGS. 43 and 44 may be used to accurately align the acetabular cup in a plane other than that influenced by the plumb bob 172. The alignment frame 176 includes a frame member 177 which defines a first end 178 and a second end 179. The fame member 177 is generally ‘L’ shaped when viewed from the side, and ‘A’ shaped when viewed in plan. An abutment pad 180 is disposed on the first end 178 and is adapted to abut a planar surface, in particular the vertical side of the operating table. For example, the surgeon may use their knee to press the abutment pad 180 against the side of the operating table. The second end 179 of the alignment frame 176 is positioned adjacent the patient's hip.

Engagement means in the form of a slotted member 181 is disposed on the second end 179 and is aligned so as to project toward the patient's hip. The open-ended slot 225 in the slotted member 181 defines a semi circular surface 226 having an internal radius of curvature which matches an external radius of curvature of the body 182 of the alignment handle 159. In other words, the slot 225 is sized so as to receive and direct the body 182 of the alignment handle 159. The geometry is such that engagement of the alignment handle 159 with the engagement means 181 forces the alignment handle 159 into a predefined orientation with respect to a second plane (different to the first plane associated with the plumb bob 172). This is because the slotted member 181 has a width 227 sufficient to ensure that when the body 182 is engaged with the slotted member 181, the longitudinal axis of the body 182 matches the axis of the slotted member 181. More particularly, when the body 182 is disposed within the slotted member 181, the body 182 is substantially perpendicular to the side of the operating table.

Preferably the hip of the patient is maintained on the operating table in a predefined position relative to the operating table, for example by using a pelvic holder 230 and/or other means for fixing the patient's position on the operating table. When each of the following conditions are met:
the patient's hip is in the predefined position on the operating table;
the first end 178 of the alignment frame 176 is abutted against the side of the operating table adjacent the patient's hip so that the slotted member 181 faces the patient's hip;
the body 182 of the alignment handle 159 is engaged with the slotted member 181; and
the rotatable needle 173 of the plumb bob 172 is aligned with the fixed needle 175 so as to indicate that the prosthetic acetabular cup is in the predefined orientation
   then the surgeon can be confident that the prosthetic acetabular cup is correctly positioned for insertion into the patient's hip.

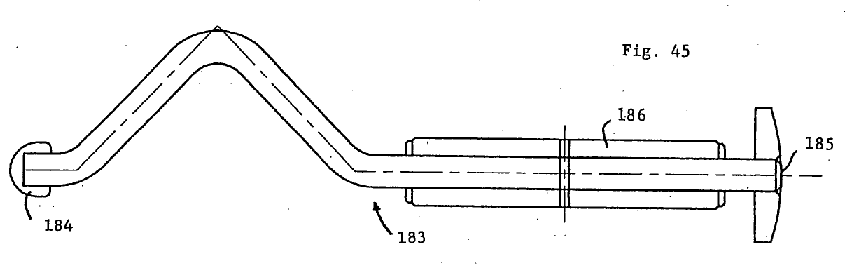

At this point the surgeon utilises the punch assembly 183, as illustrated in FIG. 45, to impact the acetabular cup into the reamed acetabulum. More particularly, the driving head 184 of the punch assembly 183 is placed against the second end 171 of the boss 170 and a hammer or similar tool us impacted against impacting surface 185 whilst the surgeon grips the punch assembly 183 via the handle 186. This process affixes the acetabular cup into the reamed acetabulum.

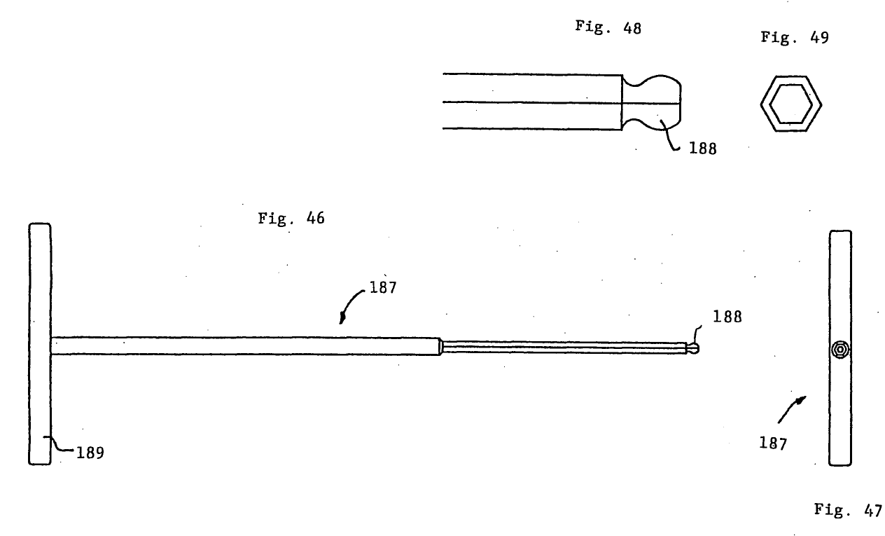

The fastener driver 187 is preferably an allen key, as illustrated in FIGS. 46 and 47, which mates with the alien socket 221 in the screw 217, 218, 219 or 220 used to affix the acetabular cup onto the cup holder 158. The allen key 187 has a rounded driving end 188, allowing the screw 217, 218, 219 or 220 to be driven from a range of angles in addition to direct alignment. This advantageously allows a degree of freedom to the surgeon to accommodate the difficultly associated with driving the screw through a wound 234 in the patient. The allen key 187 also has a handle 189 adapted for facilitating rotation of the driving head 188. Once the screw 217, 218, 219 or 220 has been disengaged from the acetabular cup, the cup holder 158 is withdrawn from the patient.

If desired, the acetabular cup can be optionally further affixed into the reamed acetabulum by fixing means such as a screw which is driven through the acetabular cup and into the patient's pelvis. A liner is then inserted into the acetabular cup and a blunt Hohmann may be inserted under the liner to avoid subsequent displacement.

The patient's leg is now held in a vertical position to allow for insertion of the stem 33, for example a C.F.P. stem, into the femoral canal. After insertion, an end 34 of the stem 33 projects from the end of the femur.

It is now necessary to determine the prosthetic neck length required for post operative correct leg length, tissue tension and muscle tension. A short trial head is placed onto the stem and the hip joint is reduced. As best shown in FIG. 70, a spacer alignment tool 189 is now assembled by attaching a left or right spacer member 190, as required, onto the alignment handle 159 in a similar manner to the way in which the cup holder 158 was previously attached to the handle assembly 159. That is, the projection 191 keys into an aperture disposed in the distal end 161 of the handle assembly 159 and the collar 163 threadedly engages the proximal end 192 of the spacer member 190. Also similar to the previous assembly of the cup alignment tool 167, the handle 162 is screwed into the spacer member at internal thread 193 provided adjacent the proximal end 192 of the spacer member 190. In other words, to assemble the spacer alignment tool 189, simply start with the cup alignment tool 167 and replace the cup holder 158 with the spacer member 190.

The spacer member 190 includes a spacer 194 which has a slot 195 adapted to engage an end 34 of the stem 33. This positions the spacer 194 intermediate the trial head and the end 34 of the femur, as shown in FIG. 70 (although the trial head is obscured within the reamed acetabulum). When properly positioned, the trial head is disposed within the hemispherical surface 203. Once again the surgeon may make use of the plumb bob 172 and the alignment frame 176 to ensure that the spacer 194 and the trial head are oriented in an anatomically correct manner whilst performing the following tests.

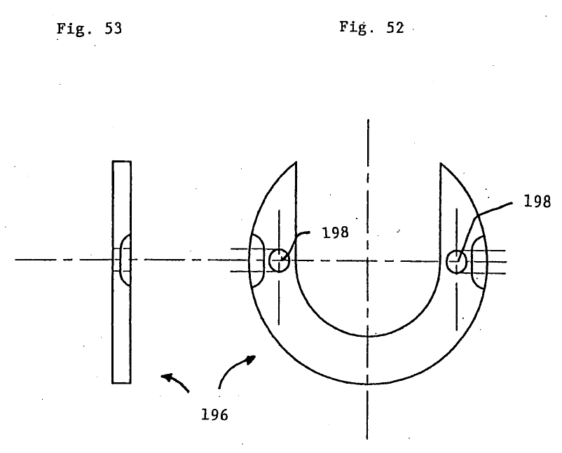

With the spacer 194 in place, the surgeon tests whether the leg length and/or tissue tension and/or muscle tension are sufficient. If not, the surgeon extracts the spacer alignment tool 189 and clips a medium width additional spacer 196 (as illustrated in FIGS. 52 and 53) onto the spacer 194. The medium width additional spacer 196 clips onto the spacer 194 via press-fitting formations 198 provided on the two components. If leg length and/or tissue tension and/or muscle tension are still insufficient, the process is again repeated with the thick width additional spacer 197 taking the place of the medium width additional spacer 196.

In other words, the testing process is repeated with spacers 194, 196 or 197 of different thicknesses (or with a different number of spacers) until the anatomically correct leg length and/or tissue tension and/or muscle tension is achieved. The surgeon then takes note of the spacer thickness (or number of spacers) which gave the best results and correlates this with the required prosthetic neck length in accordance with a predefined correlation scheme. For example, if the correct leg length and/or tissue tension and/or muscle tension is achieved without the need for an additional spacer 196 or 197, then the shortest prosthetic neck length is used. If the correct leg length, etc, is achieved with the use of the medium thickness additional spacer 196, then a medium prosthetic neck length is used. If the correct leg length, etc, is achieved with the use of the thick width additional spacer 197, then the longest prosthetic neck length is used. Of course, other correlation schemes may also be devised for other embodiments of the invention.

This process for ascertaining the prosthetic size required to give the correct leg length, etc, is preferable to the prior art method which involves repeatedly dislocating the hip joint until the correct size of prosthetic components is identified.

The hip is dislocated and the test prosthetic components are now replaced with the actual prosthetic components and the hip is reduced. The surgeon now confirms that the patient's leg has the proper range of motion and double checks that acceptable leg length, tissue tension and muscle tension have been achieved. The surgeon concludes the operation in the standard manner by re-attaching the tricipital tendon and short external rotators and closing in the various layers. The application of a cryocuff to the hip region is recommended to aid post operative rehabilitation.

The pin extractor 199 may be utilised if it is desired to salvage the pins 143 which were previously driven into the femoral head 3 and remain embedded within the femoral head 3 once it has been removed from the patient. Also, it may be necessary to conduct tests on the femoral head 3, in which case removal of the pins 143 may be required. With the tongs 200 and 201 radially separated, a pin 143 may be inserted into aperture 202, past engagement member 203. The tongs are then squeezed radially together such that the engagement member 203 exerts a transverse force onto the notches 228 disposed adjacent the end 229 of the pin 143, thereby causing the pin extractor 199 to grip the pin 143 tightly. A longitudinal force is then exerted upon the pin extractor 199 which is transferred to the pin 143 to assist in extraction of the pin 143 from the severed femoral head 3. The longitudinal force may be supplied by impacting a hammer onto projection 204.

It will be appreciated that the preferred method for hip replacement surgery provides a number of advantages in comparison to the prior art methods. In particular, the hip joint does not require dislocation prior to the oesteotomy, the accuracy and consistency of the reaming of the femoral canal and acetabulum is improved and the process for establishing the required sizing and orientation of prosthetic components generally entails less trial and error. Typically this results in a surgical procedure in which the paiient's ligaments and muscular attachments experience minimal disturbance, allowing for more rapid patient rehabilitation with less postoperative pain. Further advantages typically associated with the preferred embodiment include reduced scarring, reduced blood loss and clotting, reduced risk of infection, shorter hospital admittance and reduced costs.

Although the invention has been described with reference to specific examples, it will be appreciated by those skilled in the art that the invention may be embodied in many other forms.

1-100. (Cancelled)

101. A surgical jig for application to a bone, said jig comprising:
jig location means adapted to mate with a bone formation so as to locate the jig in a predefined relationship with the bone; and
surgical tool guide means disposed relative to said jig location means such that, when said jig is mated with said bone formation, the surgical tool guide means is adapted to guide a surgical tool towards a predefined site on said bone formation.

102. A surgical jig according to claim 101 wherein said jig location means includes a spacer adapted to abut said bone formation so as to position the surgical tool guide means in a predefined distance from said bone formation.

103. The surgical jig according to claim 102 wherein said spacer is an elongate rod.

104. The surgical jig according to claim 101 wherein said jig location means includes a surface adapted to receive said bone formation.

105. The surgical jig according to claim 104 wherein said surface includes contours which are adapted to mate with contours of the bone.

106. The surgical jig according to claim 101 wherein said jig further includes a head disposed on a distal end of a handle.

107. The surgical jig according to claim 103 wherein said jig further includes a head disposed on a distal end of a handle, said rod being disposed on said head.

108. The surgical jig according to claim 106 wherein said jig location means includes a surface adapted to receive said bone formation and wherein said surface includes contours which are adapted to mate with contours of the bone, said contours being provided upon a base of said head.

109. The surgical jig according to claim 101 wherein said surgical tool guide means is provided by at least one slot.

110. The surgical jig according to claim 109 wherein said jig further includes a head disposed on a distal end of a handle, said at least one slot being disposed upon said head.

111. The surgical jig according to claim 110 wherein said spacer is an elongate rod, said rod being disposed on said head and wherein an elongate dimension of said rod extends at an acute angle relative to an elongate dimension of the at least one slot.

112. The surgical jig according to claim 109 wherein said at least one slot is adapted to receive and guide a reciprocating blade.

113. The surgical jig according to claim 110 wherein more than one slot is provided upon said head, each slot being disposed at a different lateral position so as to allow the slots to guide a surgical tool to more than one predefined site on said bone formation.

114. The surgical jig according to claim 113 wherein each of said predefined sites on the bone formation are selected so as to correspond with different sized prosthetics.

115. The surgical jig according to claim 101 further comprising fixing means for securing the jig to the bone in a required position.

116. The surgical jig according to claim 115 wherein said jig further includes a head disposed on a distal end of a handle and said fixing means include pins drivable through holes in the head of the jig and into the bone.

117. The surgical jig according to claim 101 wherein said jig is adapted to mate with a femur.

118. The surgical jig according to claim 103 wherein said jig is adapted to mate with a femur and wherein said elongate rod has an end adapted to abut a trochanteric fossa defined by said femur.

119. The surgical jig according to claim 118 wherein said rod is sized so as to position said surgical tool guide means at a distance of between approximately 5 mm to 25 mm from said trochanteric fossa.

120. The surgical jig according to claim 119 wherein said distance is approximately 15 mm.

121. The surgical jig according to claim 101 wherein said jig location means includes first and second opposed lugs disposed on a body of said jig.

122. The surgical jig according to claim 121 wherein said lugs are adapted to mate with a neck of a femur.

123. The surgical jig according to claim 122 wherein each of said lugs have an elongate dimension oriented such that, when said jig is mated with said bone formation, the elongate dimension of the lugs is oriented in substantially the same direction as an elongate dimension of the neck of the femur.

124. The surgical jig according to claim 121 wherein said lugs are snap fittingly engageable with said bone formation.

125. The surgical jig according to claim 123 wherein said lugs are arcuate with a centre of curvature extending along the elongate dimension.

126. The surgical jig according to claim 125 wherein said jig location means includes a surface adapted to receive said bone formation and said surface defines an inverted V-shape.

127. The surgical jig according to claim 126 wherein said inverted V-shaped surface is provided by an inverted V-shaped member disposed on said body.

128. The surgical jig according to claim 127 wherein said jig location means includes first and second opposed lugs disposed on a body of said jig, said inverted V-shaped member being laterally disposed from said lugs.

129. The surgical jig according to claim 128 wherein said surgical tool guide means is disposed intermediate said inverted V-shaped member and said lugs.

130. The surgical jig according to claim 106 wherein said handle has a length sufficient to extend externally of a patient when the head of the jig is mated to the bone formation.

131. The surgical jig according to claim 121 wherein said jig location means includes a side wall of said body shaped so as to mate with said bone formation.

132. The surgical jig according to claim 131 wherein said side wall is arcuate.

133. The surgical jig according to claim 132 wherein said arcuate side wall is shaped so as to abut a natural curvature of the greater trochanter in the femur.

134. A surgical jig for application to a femur during hip replacement surgery, said jig comprising:
at least one lug adapted to mate with a bone formation on said femur so as to locate the jig in a predefined relationship with the femur; and
at least one slot disposed relative to said at least one lug such that, when said jig is mated with said femur, the at least one slot is adapted to guide a blade towards a predefined site on said bone formation.

135. The surgical jig according to claim 134 wherein said at least one lug is a rod disposed on a head of the jig so as to define an end adapted to engage a femoral bone formation, thereby positioning said at least one slot in a predefined relationship to said bone formation on said femur.

136. The surgical jig according to claim 135 further including contours disposed on a base of said head, said contours matching, and being adapted to mate with the bone formation on said femur.

137. A method of performing hip replacement surgery on a patient, said method comprising the steps of:
a) exposing a first femoral bone formation and a second femoral bone formation of the patient;
b) applying a jig having surgical tool guide means to the first femoral bone formation such that said surgical tool guide means is proximal to the second femoral bone formation;
c) using the surgical tool guide means to guide a cutting tool so as to osteotomise the second bone formation; and
d) removing a severed femoral head from the patient's hip joint.

138. The method according to claim 137 wherein the first femoral bone formation is a greater trochanter and the second femoral bone formation is a femoral neck.

139. The method according to claim 137 wherein step a) includes making an initial incision in a thigh of the patient of between 3 cm and 7 cm in length.

140. The method according to claim 139 wherein one end of said incision is approximately 1 cm to 3 cm behind a greater trochanter and wherein said incision curves backwards from said end.

141. The method according to claim 137 wherein step a) includes the step of exposing a posterial aspect of a femoral neck by detaching a tricipital tendon of the patient.

142. The method according to claim 141 wherein said tricipital tendon is tagged for later re-attachment.

143. The method according to claim 137 wherein step a) includes the step of performing a capsulectomy to expose a femoral neck.

144. The method according to claim 137 in which a leg of the patient is maintained in a position approximately 45° from vertical during at least some of the surgical steps.

145. The method according to claim 144 wherein said position of said leg is maintained by a leg holder.

146. The method according to claim 137 wherein the jig in step b) is a surgical jig for application to a bone, said jig comprising:
jig location means adapted to mate with a bone formation so as to locate the jig in a predefined relationship with the bone; and
surgical tool guide means disposed relative to said jig location means such that, when said jig is mated with said bone formation, the surgical tool guide means is adapted to guide a surgical tool towards a predefined site on said bone formation.

147. The method according to claim 138 wherein, when said jig is applied to the femur, the surgical tool guide means is disposed approximately at a predefined distance from the base of the greater trochanter.

148. The method according to claim 147 wherein said predefined distance is between 10 mm and 20 mm.

149. The method according to claim 137 further comprising the step of forming a notch in a femoral neck on the femoral head side of the osteotomological cut.

150. The method according to claim 137 further comprising the steps of:
e) placing a guide wire into a femoral canal of a femoral shaft of the patient;
f) using the guide wire to guide a flexible reamer so as to bore an aperture into said femoral canal; and
g) inserting a prosthetic stem into said aperture such that a first end of the stem protrudes from an end of the femoral shaft.

151. The method according to claim 150 further comprising the steps of:
h) placing said first end into an acetabulum of the patient;
i) inserting spacers intermediate the end of the femoral shaft and the acetabulum;
j) using a spacer to determine reference points on an acetabulum of the patient; and
k) using said reference points to provide guidance for reaming of the acetabulum.

152. The method according to claim 151 further comprising the steps of:
l) placing a trial prosthetic head onto said first end;
m) placing a trial acetabular cup into the reamed actabulum;
n) placing said trial prosthetic head into said trial acetabular cup;
o) inserting spacers intermediate the end of the femoral shaft and the trial acetabular cup until a required fit between the trial prosthetic head and the trial acetabular cup is obtained; and
p) correlating the number or size of spacers with a required size of a prosthetic component.

153. A method of using a jig according to claim 101 for placement on a femur for guiding cutting of the femur in a method of hip replacement.

154. A method of using a jig for assembly of a guidance system for use in a method of hip replacement surgery.

155. A method of performing hip replacement surgery on a patient, said method comprising the steps of:
i) exposing a greater trochanter, and a femoral neck of the patient;
ii) osteotomising the femoral head;
iii) inserting a prosthetic stem or trial stem into a femoral shaft of the patient such that a first end of the stem protrudes beyond an end of the femoral shaft;
iv) positioning the first end into an acetabulum of the patient;
v) applying one or more spacers to the first end of the stem;
vi) using said one or more spacers to determine reference positions on the acetabulum; and
vii) using said reference positions to guide reaming of said acetabulum.

156. The method according to claim 155 wherein said spacers include markings adapted to indicate said reference positions.

157. The method according to claim 155 wherein step vii) includes determining a centre of said reference positions and using said centre as a centre of reaming.

158. A method of performing hip replacement surgery on a patient, said method comprising the steps of:
A) exposing a greater trochanter, and a femoral neck of the patient;
B) osteotomising the femoral head;
C) inserting a prosthetic stem or trial stem into a femoral shaft of the patient such that a first end of the stem protrudes beyond an end of the femoral shaft;
D) inserting a prosthetic acetabular cup or trial acetabular cup into a reamed acetabulum of the patient;
E) applying one or more spacers to the first end of the stem until a desired tissue tension in the patient's hip joint region has been obtained; and
F) correlating the number or size of spacers with a required prosthetic component size.

159. The method according to claim 158 wherein said required prosthetic component size relates to a neck length of a prosthetic femoral neck and head component.

160. A method of performing hip replacement surgery on a patient, said method comprising the steps of:
A) exposing a greater trochanter, and a femoral neck of the patient;
B) osteotomising the femoral head;
C) inserting a prosthetic stem or trial stem into a femoral shaft of the patient such that a first end of the stem protrudes beyond an end of the femoral shaft;
D) inserting a prosthetic acetabular cup or trial acetabular cup into a reamed acetabulum of the patient; and
E) applying one or more spacers to the first end of the stem so as to determine a required orientation of the acetabular cup relative to the stem.

161. A spacer for use in hip replacement surgery, said spacer comprising:
a handle defining a first end; and
alignment means disposed at or adjacent to said first end, said alignment means being adapted to mate with a prosthetic acetabular cup such that, in use, the cup is positioned in a predefined relationship with reference to an acetabulum of a patient.

162. The spacer according to claim 161 wherein said alignment means defines a plane adapted to mate with a corresponding plane of the prosthetic acetabular cup.

163. The spacer according to claim 162 wherein said plane forms an acute angle with reference to a plane defined by said handle.

164. The spacer according to claim 163 wherein said angle is within the range of 5° to 25°.

165. The spacer according to claim 164 wherein said angle is within the range of 10° to 20°.

166. The spacer according to claim 165 wherein said angle is approximately 15°.

167. The spacer according to claim 162 wherein said plane is defined by a semi-circular surface.

168. The spacer according to claim 167 wherein said alignment means includes a slot adapted to receive a protruding end of a prosthetic femoral stem.

169. The spacer according to claim 161 further comprising orientation guidance means adapted to provide guidance as to whether the alignment means is positioned in said predefined relationship with reference to an acetabulum of a patient.

170. The spacer according to claim 169 wherein said orientation guidance means includes a plumb bob rotatably disposed upon said spacer so as to hang substantially vertically under the influence of gravity.

171. The spacer according to claim 170 further including a reference point disposed on said spacer such that the plumb bob hangs in alignment with the reference point when the alignment means is positioned in said predefined relationship with reference to an acetabulum of a patient.

172. The spacer according to claim 161 wherein said alignment means includes engagement means adapted to engage the prosthetic acetabular cup.

173. The spacer according to claim 172 wherein said engagement means includes at least one lug adapted to mate with an internal surface of said cup.

174. The spacer according to claim 161 wherein said alignment means defines an outer circumferential edge having a radius of curvature substantially corresponding to a radius of curvature of the prosthetic acetabular cup.

175. An alignment handle for use in hip replacement surgery, said alignment handle comprising:
a body defining a distal end and a proximal end;
a gauge disposed at or proximate to said proximal end; and
a connector disposed at or proximate to said distal end for connection of a prosthetic acetabular cup,
said gauge being adapted to indicate whether said prosthetic acetabular cup is in a predefined orientation with respect to at least a first plane in a referential system.

176. The alignment handle according to claim 175 wherein said referential system includes a local gravitational field.

177. The alignment handle according to claim 176 wherein said gauge includes a plumb bob rotatably disposed upon said body so as to hang substantially vertically under the influence of said local gravitational field.

178. The alignment handle according to claim 177 further comprising a reference point disposed on said body such that the plumb bob hangs in alignment with the reference point when said prosthetic acetabular cup is in said predefined orientation with respect to said first plane.

179. The alignment handle according to claim 175 further comprising a handle disposed intermediate said distal end and said proximal end.

180. An alignment frame adapted for use with an alignment handle according to claim 175, said alignment frame comprising:
a first end adapted for abutment against a planar surface so as to align a second end of said alignment frame with respect to said planar surface; and
engagement means disposed on said second end and adapted for engagement with said alignment handle.

181. The alignment frame according to claim 180 wherein said engagement means includes an open-ended slot.

182. The alignment frame according to claim 181 wherein said open-ended slot defines a semi-circular surface having an internal radius matching an external radius of the body of said alignment handle.

183. The alignment frame according to claim 181 wherein said engagement means has a geometry such that engagement of the alignment handle with the engagement means forces the alignment handle to adopt a predefined orientation with respect to the alignment frame.

184. The alignment frame according to claim 180 wherein engagement of the alignment handle into the engagement means forces the alignment handle into a predefined orientation with respect to at least a second plane different to said first plane.

185. The alignment frame according to claim 180 further comprising a predefined patient hip position relative to an operating table such that when each of:
the patient's hip is in the predefined position;
the first end of the alignment frame is abutted against a planar surface of the operating table;
the body of the alignment handle is engaged with the engagement means of the alignment frame; and
the gauge of the alignment handle indicates that the prosthetic acetabular cup is in the predefined orientation,
then the prosthetic acetabular cup is positioned for insertion into the patient's hip.

186. The alignment frame according to claim 185 wherein said planar surface of the operating table is a side of the operating table and wherein the second end is positioned adjacent the patient's hip.

187. A method for extracting an osteotomitised femoral head from a patient's hip joint, said method comprising the following steps:
a) drilling a hole into the femoral head,
b) threading a self-tapping threaded member into said hole; and
c) exerting an outwardly directed force onto said threaded member which is, in turn, exerted onto the femoral head.

188. A method for extracting an osteotomitised femoral head from a patient's hip joint, said method comprising the step of wedging an end of a scoop under the femoral head and using said scoop as a lever so as to exert an outwardly directed force onto said femoral head.

189. A method for inserting a prosthetic acetabular cup into a patient's reamed acetabulum, said method comprising the steps of:
a) mounting said prosthetic acetabular cup onto a cup alignment tool;
b) manipulating the cup alignment tool such that the prosthetic acetabular cup is adjacent the reamed acetabulum;
c) orienting the cup alignment tool to ensure that the prosthetic acetabular cup is in an anatomically correct orientation for insertion into the reamed acetabulum; and
d) impacting the prosthetic acetabular cup into the reamed acetabulum.

190. The method according to claim 189 wherein said cup alignment tool includes an alignment handle for use in hip replacement surgery, said alignment handle comprising:
a body defining a distal end and a proximal end;
a gauge disposed at or proximate to said proximal end; and
a connector disposed at or proximate to said distal end for connection of a prosthetic acetabular cup,
said gauge being adapted to indicate whether said prosthetic acetabular cup is in a predefined orientation with respect to at least a first plane in a referential system and wherein said gauge is used in step c) to determine whether said prosthetic acetabular cup is in said anatomically correct orientation with respect to a first plane.

191. The method according to claim 190 wherein step c) further comprises the use of an alignment frame adapted for use with an alignment handle for use in hip replacement surgery, said alignment handle comprising:
a body defining a distal end and a proximal end;
a gauge disposed at or proximate to said proximal end; and
a connector disposed at or proximate to said distal end for connection of a prosthetic acetabular cup,
said gauge being adapted to indicate whether said prosthetic acetabular cup is in a predefined orientation with respect to at least a first plane in a referential system, said alignment frame comprising:
a first end adapted for abutment against a planar surface so as to align a second end of said alignment frame with respect to said planar surface; and
engagement means disposed on said second end and adapted for engagement with said alignment handle, wherein engagement of the alignment handle into the engagement means forces the alignment handle into a predefined orientation with respect to at least a second plane different to said first plane, to orient the prosthetic acetabular cup into said anatomically correct orientation with respect to a second plane different to said first plane.

192. The method according to claim 191 wherein step c) comprises the following sub-steps:
c1) positioning the patient's hip is in a predefined position on an operating table;
c2) abutting the first end of the alignment frame against a planar surface of the operating table;
c3) engaging the body of the alignment handle with the engagement means of the alignment frame; and
c4) using the gauge of the alignment handle to indicate whether the prosthetic acetabular cup is in said anatomically correct orientation.

193. A method for ascertaining a size of a prosthetic neck length during hip replacement surgery, said method comprising the steps of:
a) installing a prosthetic stem into a patient's femur such that an end of said stem projects from an end of the femur;
b) placing a trial head onto the end of the prosthetic stem;
c) using a spacer alignment tool to position a spacer intermediate the trial head and the end of the femur;
d) orienting the spacer alignment tool to ensure that the trial head is in an anatomically correct orientation;
e) testing whether one or more of the patient's leg length, tissue tension and muscle tension is anatomically correct;
f) repeating steps c), d) and e) with different spacer thicknesses or numbers of spacers until one or more of the patient's leg length, tissue tension and muscle tension is anatomically acceptable and taking note of the spacer thickness or number of spacers which gives the best result; and
g) correlating the spacer thickness or number of spacers determined in step f) with the required prosthetic neck length in accordance with a predefined correlation scheme.
